(19) United States
(12) Patent Application Publication
(10) Pub. No.: US 2019/0021891 A1
(43) Pub. Date: Jan. 24, 2019

(54) DEVICES AND METHODS FOR GASTROINTESTINAL BYPASS

(71) Applicant: VALENTX, INC., Maple Grove, MN (US)

(72) Inventors: Roland MAUDE-GRIFFIN, Edina, MN (US); Jon ST. GERMAIN, Elk River, MN (US); Cole CHEN, Maple Grove, MN (US)

(21) Appl. No.: 15/983,082

(22) Filed: May 17, 2018

Related U.S. Application Data
(60) Provisional application No. 62/507,775, filed on May 17, 2017.
(60) Provisional application No. 62/523,801, filed on Jun. 23, 2017.
(60) Provisional application No. 62/612,600, filed on Dec. 31, 2017.

(51) Int. Cl. A61F 5/00; A61B 17/04; A61B 90/00; A61F 2/04

(52) U.S. Cl. CPC A61F 5/0076; A61B 17/0401; A61B 90/39; A61B 2017/00818; A61F 5/005; A61F 2/04; A61F 5/0089; A61F 5/0036

(57) ABSTRACT

Gastrointestinal bypass devices are described. A gastrointestinal bypass device may comprise a cuff configured to be placed at least partially in an esophagus of the patient, and a sleeve configured to be placed at least partially in a stomach of the patient. In one embodiment, the gastrointestinal bypass device may also comprise a tether coupled to the cuff and/or the sleeve. The tether may be configured to be placed through a fistula between an esophagus and a skin of the patient. The tether may have a length sufficient to extend from the cuff and/or the sleeve to the skin.

### CROSS-REFERENCE TO RELATED APPLICATIONS

This application claims the benefit of U.S. provisional application No. 62/507,775, filed May 17, 2017; 62/523,801, filed Jun. 23, 2017; and 62/612,600, filed Dec. 31, 2017. These applications are hereby incorporated by reference in their entireties.

### BACKGROUND

Diabetes, heart disease, and other obesity-related conditions may be treated surgically with bariatric procedures such as jejuno-ileal bypass, jejuno-colic bypass, biliopancreatic diversion, gastric bypass, vertical sleeve gastrectomy, adjustable gastric banding, and gastroplasty. These procedures may be effective for weight control and treatment of chronic conditions. However, these procedures carry with them substantial shortcomings, including the risk of infection and other risks accompanying surgery. Some of these procedures induce radical permanent changes to the gastrointestinal anatomy, thus foreclosing subsequent surgical intervention.

What is needed are devices and methods that use non-surgical techniques that avoid the risks associated with gastrointestinal bypass surgery. What is also needed are devices and methods for gastrointestinal bypass that allow for additional or revision procedures to be performed. What is also needed are devices and methods for gastrointestinal bypass that are reversible.

### SUMMARY

Gastrointestinal bypass devices are described. A gastrointestinal bypass device may comprise a cuff configured to be placed at least partially in an esophagus of the patient, and a sleeve configured to be placed at least partially in a stomach of the patient. In one embodiment, the gastrointestinal bypass device may also comprise a tether coupled to the cuff and/or the sleeve. The tether may be configured to be placed through a fistula between an esophagus and a skin of the patient. The tether may have a length sufficient to extend from the cuff and/or the sleeve to the skin.

In another embodiment, the gastrointestinal bypass device may also comprise a gastric retainer coupled to the sleeve. The gastric retainer may be configured to be placed at least partially in the stomach. The gastric retainer may have a collapsed configuration and a deployed configuration. The gastric retainer in the collapsed configuration may have a size small enough to pass through the esophagus. The gastric retainer in the deployed configuration may have a size too large to pass through the esophagus.

Methods for delivering a gastrointestinal bypass device are also described. In one embodiment, a method may comprise placing a cuff at least partially in an esophagus of a patient, placing a sleeve at least partially in a stomach of the patient, creating a fistula between the esophagus and a skin of the patient, placing a tether through the fistula, and securing the tether to an anchor site.

### BRIEF DESCRIPTION OF THE DRAWINGS

FIGS. 1A-1F show various embodiments of a gastrointestinal bypass device 1000.

FIGS. 2A-2J show various embodiments of a gastric retainer 1130 including a ring 1131.

FIG. 3 shows one embodiment of a gastric retainer 1130 including a balloon 1132.

FIGS. 4A-4C show two embodiments of a gastric retainer 1130 including arms 1133.

FIGS. 5A-5G show various embodiments of a tether 1310.

FIGS. 6A-6B show a tether 1310 with intraesophageal portion 1310in extended distally and proximally, respectively. FIGS. 6C-6D show one embodiment of a tether 1310 including a stiffened portion 1310a. FIG. 6E shows another embodiment of a tether 1310 including a stop 1318.

FIGS. 7A-7E show various embodiments of a bridle 1320.

FIGS. 8A-8C show various embodiments of an elongation element 1330.

FIGS. 9A-9B show two embodiments of a pull tab 1340.

FIGS. 10A-10B shows one embodiment of an access port 1350.

FIG. 11 shows one embodiment of sensors 1360.

FIG. 12 shows one embodiment of electrodes 1370.

FIG. 13A-13C show various embodiments of markings 1380.

FIGS. 14A-14D show two embodiments of a tether anchor 1400 including a skin button 1410.

FIGS. 15A-15B show another embodiment of a tether anchor 1400 including a skin button 1420.

FIGS. 16A-16B show two embodiments of a barbed suture 1430.

FIGS. 17A-17H show various embodiments of a tether anchor 1400 including at least one barbed suture 1430.

FIGS. 18A-18B show two embodiments of tether anchor 1400 including a fistula anchor 1440.

FIGS. 19A-19B show tether anchor 1400 including a fistula anchor 1440 placed in a fistula.

FIG. 20 shows one embodiment of a tether anchor 1400 including a bone attachment 1450.

FIG. 21 shows one embodiment of a tether anchor 1400 including a tooth attachment 1460.

FIGS. 22A-22B show various embodiments of a tether anchor 1400 including a nasal septum attachment 1470.

FIG. 23 shows one embodiment of a sleeve delivery device 1500.

FIGS. 24A-24H show one embodiment of a method for delivering cuff-sleeve assembly 1100.

FIGS. 25A-25B show two embodiment of a balloon catheter 1610 and 2610.

FIG. 26 shows one embodiment of a bite block 1620.

FIGS. 27A-27B show one embodiment of a balloon depth arm 1630.

FIGS. 28A-28B show one embodiment of a needle guide 1730.

FIG. 29 shows one embodiment of a guidewire holder 1740.

FIG. 30A shows one embodiment of a dilator 1750. FIG. 30B shows one embodiment of a sheath 1760. FIG. 30C shows dilator 1750 inside sheath 1760. FIG. 30D shows another embodiment of a sheath 1760.

FIGS. 31A-31F show one embodiment of a method for placing tether 1310.

FIGS. 32A-32G show another embodiment of a method for placing tether 1310.

FIGS. 33A-33E show another embodiment of a method for placing tether 1310.

FIGS. 34A-34C show various embodiments of a temporary tube 1780.

FIGS. 35A-35C show one embodiment of a method for removing cuff-sleeve assembly 1100.

FIG. 36 shows one embodiment of a flow restriction device 2000.

FIG. 37 shows one embodiment of an esophageal stent 3100.

### DESCRIPTION

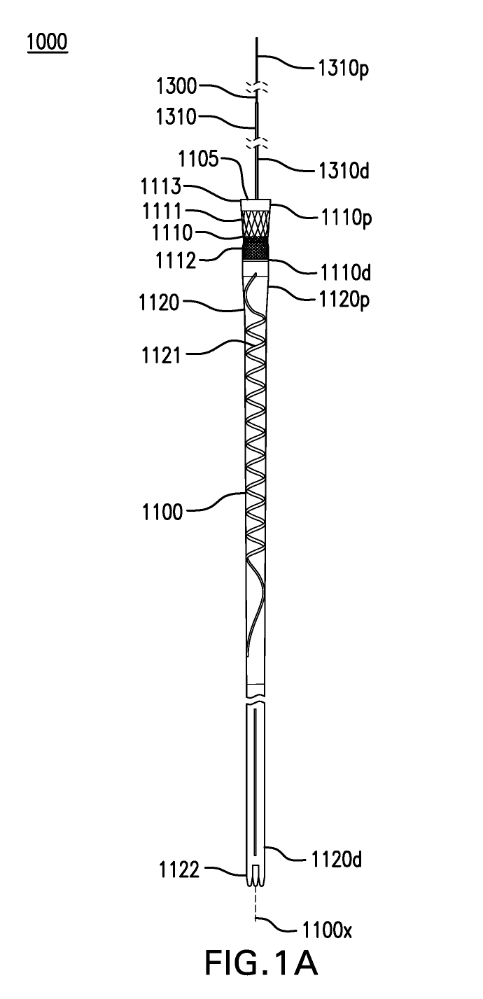

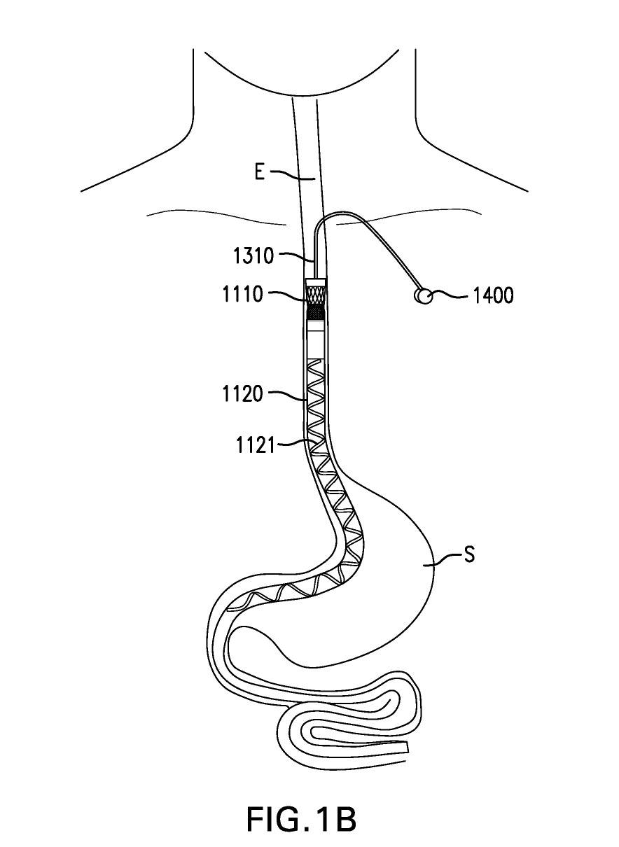

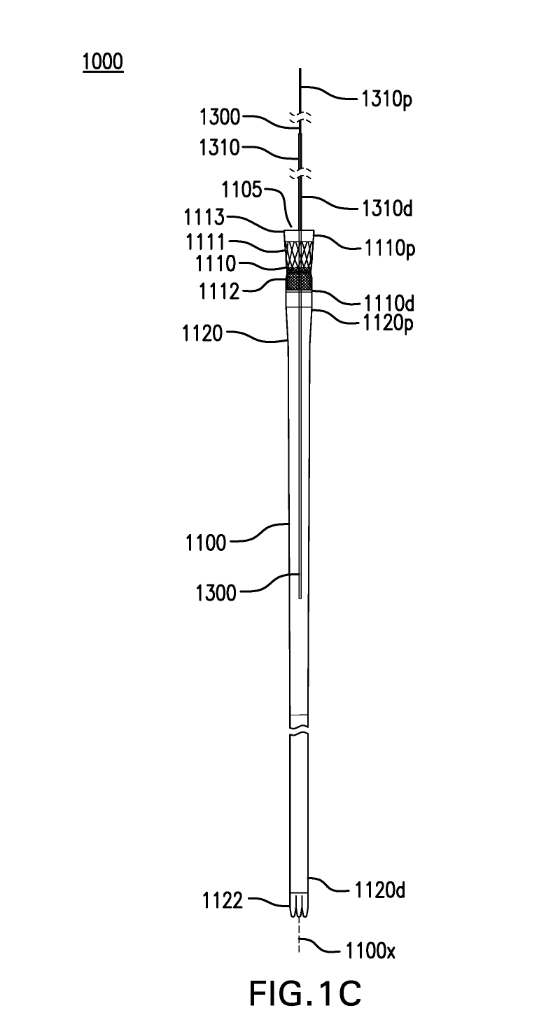

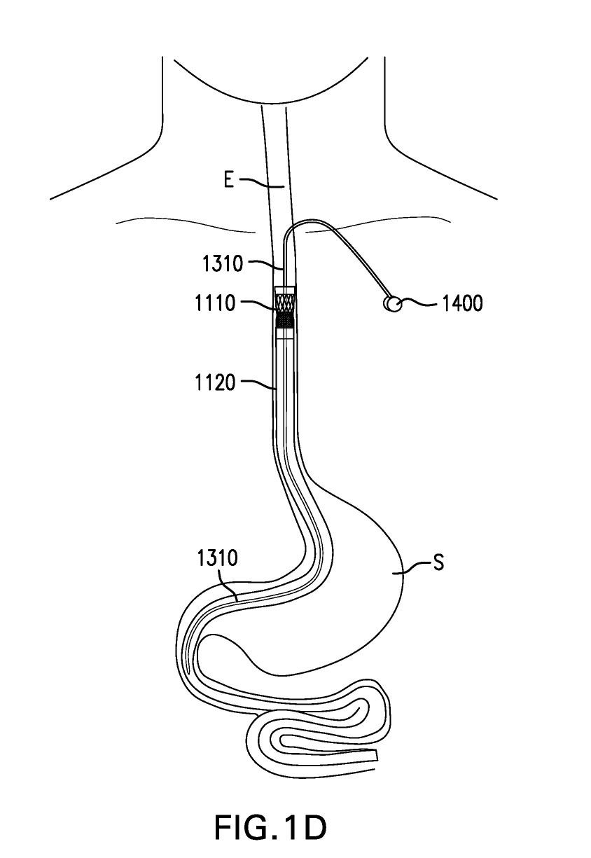

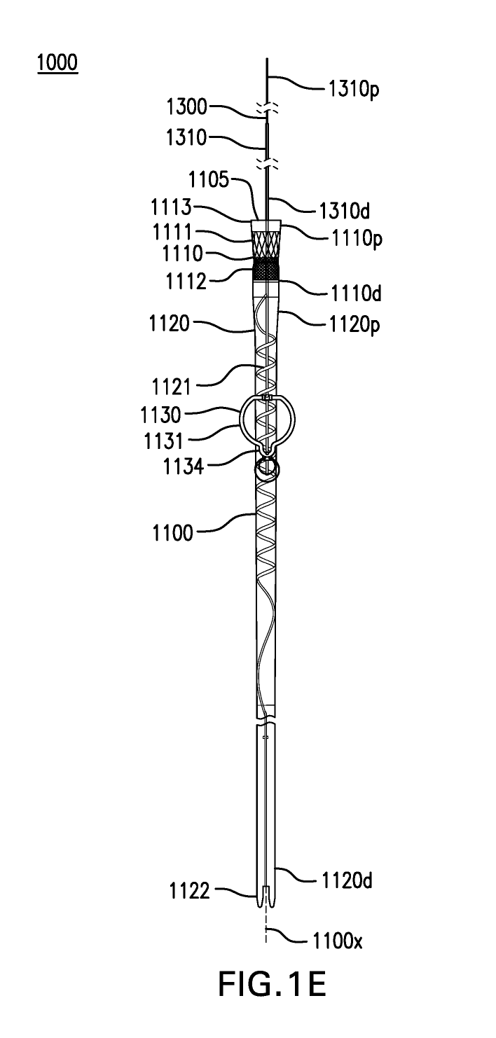

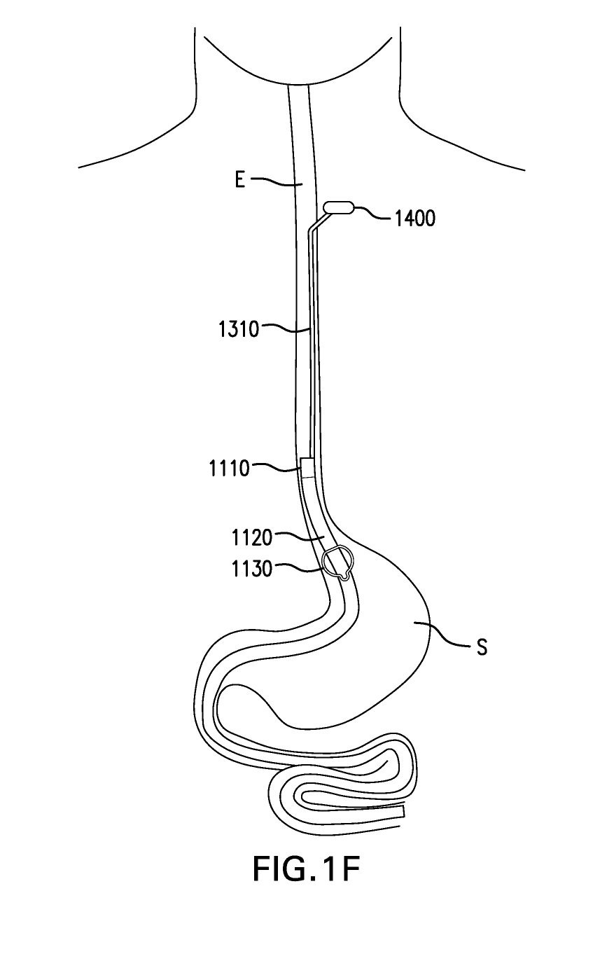

FIGS. 1A-1F show various embodiments of a gastrointestinal bypass device 1000. Gastrointestinal bypass device 1000 may allow food from the esophagus to bypass the stomach and part of the intestine.

Gastrointestinal bypass device 1000 may include a cuff-sleeve assembly 1100. Cuff-sleeve assembly 1100 may include a longitudinal axis 1100x. Cuff-sleeve assembly 1100 may include a lumen 1105. Cuff-sleeve assembly 1100 may be configured to be placed in one or more of the esophagus, stomach, and intestine.

Cuff-sleeve assembly 1100 may include a cuff 1110. Cuff 1110 may include a proximal portion 1110p and a distal portion 1110d.

Cuff 1110 may be configured to be placed at least partially in the esophagus. Proximal portion 1110p of cuff 1110 may be configured to be placed above the gastroesophageal junction (GEJ). Proximal portion 1110p of cuff 1110 may be configured to be placed about 8 cm to about 15 cm above the gastroesophageal junction (GEJ), about 10 cm to about 12 cm above the GEJ, or about or at least about 1, 2, 3, 4, 5, 6, 7, 8, 9, 10, 11, 12, 13, 14, 15, 16, 17, 18, 19, 20, 21, 22, 23, 24, 25, or more cm above the GEJ, and ranges including any two of the aforementioned values.

Cuff 1110 may direct food from the esophagus into lumen 1105. Cuff 1110 may conform to an inside of the esophageal wall. Cuff 1110 may maintain contact with an inside of the esophageal wall. Cuff 1110 may expand and collapse with an inside of the esophageal wall. Cuff 1110 may be able to move in the esophagus, such as with swallowing and changes in posture.

Cuff 1110 may be tubular. Cuff 1110 may have a length of about 30 mm to about 45 mm. Cuff 1110 may have a width and/or thickness that is uniform or varying along its length. Cuff 1110 may have a width of about 20 mm to about 30 mm. Cuff 1110 may have a thickness of about 0.1 mm to about 0.5 mm. Cuff 1110 may include one or more layers. Cuff 1110 may be flexible and compliant. Cuff 1110 may be made of one or more of a polyurethane elastomer, silicone, and any other suitable material. Cuff 1110 may be at least partially made of PELLETHANE®.

Cuff 1110 may include a stent 1111. Stent 1111 may be coupled to proximal portion 1110p of cuff 1110. Stent 1111 may be coupled to an inside, outside, or between layers of cuff 1110. Stent 1111 may be made of one or more of a metal, a polymer, and any other suitable material. Stent 1111 may be at least partially made of nitinol. Stent 1111 may have a length of about 30 mm. Stent 1111 may provide an outward bias which is large enough to allow an outside of cuff 1110 to maintain contact with the inside of the esophageal wall. Stent 1111 may provide an outward bias which is not so large as to permanently stretch the esophagus.

Cuff 1110 may include a braid 1112. Braid 1112 may be coupled to cuff 1110. Braid 1112 may be coupled to an inside, outside, or between layers of cuff 1110. Braid 1112 may be distal to stent 1111. Braid 1112 may partially overlap stent 1111. Braid 1112 may have a length of about 20 mm. Braid 1112 may have a width of about 20 mm to about 25 mm. Braid 1112 may be made of one or more of a metal, a polymer, and any other suitable material. Braid 1112 may be at least partially made of PEEK. Braid 1112 may provide additional hoop strength to a distal portion of stent 1111. Braid 1112 may reduce a likelihood of cuff 1110 tipping in the esophagus E.

Cuff 1110 may include a drawstring 1113. Drawstring 1113 may be coupled to proximal portion 1110p of cuff 1110.

Cuff 1110 may include one or more cuffs described in U.S. Pat. Nos. 7,837,669, 7,881,797, 8,070,743, 8,182,441, 8,182,459, 9,060,844, 9,675,489, and 9,757,264; and U.S. Patent Application Publication Nos. 2005/0177181, 2006/0155375, 2009/0012544, 2009/0012541, 2014/0188245, 2014/0207159, and 2016/0193063, which are incorporated by reference in their entireties.

Cuff-sleeve assembly 1100 may include a sleeve 1120. Sleeve 1120 may include a proximal portion 1120p and a distal portion 1120d. Proximal portion 1120p of sleeve 1120 may be coupled to distal portion 1110d of cuff 1110. Sleeve 1120 may be formed together with or separately from cuff 1110. Sleeve 1120 and cuff 1110 may be detachable from each other (e.g., via complementary coupling mechanisms), or integral and non-detachable. In some cases, cuff 1110 may be directly attached to sleeve 1120 either through mechanical attachment, thermal fusing, or directly laminated into sleeve 1120.

Sleeve 1120 may be configured to be placed in one or more of the esophagus, stomach, and intestine. Sleeve 1120 may direct food passing through cuff 1110 to bypass the stomach and/or part of the intestine. Sleeve 1120 may direct food passing through cuff 1110 into the intestine, such as one or more of the duodenum, jejunum, and any other part of the intestine.

Sleeve 1120 may be tubular. Sleeve 1120 may have a length of about 60 cm to about 200 cm. Sleeve 1120 may have a width and/or thickness that is uniform or varying along its length. Sleeve 1120 may have a width of about 15 mm to about 25 mm. Sleeve 1120 may have a thickness of about 0.1 mm to about 0.5 mm. Sleeve 1120 may be flexible and compliant. Sleeve 1120 may be floppy or flaccid. Sleeve 1120 may allow peristaltic forces to act on its contents. Sleeve 1120 may be collapsible to allow the lower esophageal sphincter (LES) to close substantially normally. Sleeve 1120 may not stretch substantially outwardly and/or longitudinally. Sleeve 1120 may be made of one or more of a polyurethane elastomer, silicone, and any other suitable material. Sleeve 1120 may be at least partially made of PELLETHANE®. Sleeve 1120 may include one or more layers.

Sleeve 1120 may include an anti-kinking stripe 1121. Anti-kinking stripe 1121 may reduce kinking and/or twisting of sleeve 1120. Anti-kinking stripe 1121 may be coupled to an inside, outside, or between layers of sleeve 1120. Anti-kinking stripe 1121 may run at least part of the length of sleeve 1120. Anti-kinking stripe 1121 may have a width and/or thickness that is uniform or varying along its length. Anti-kinking stripe 1121 may have a width of about 2 mm to about 4 mm. Anti-kinking stripe 1121 may have a thickness of about 1 mm. Anti-kinking stripe 1121 may be at least partially made of a polymer such as PELLETHANE® mixed with barium sulfate. Anti-kinking stripe 1121 may be radiopaque.

Anti-kinking stripe 1121 may have one or more of a spiral or helical configuration, straight or longitudinal configuration, circumferential configuration, and any other suitable configuration. Anti-kinking stripe 1121 may have a spiral configuration along at least a portion of sleeve 1120. Anti-kinking stripe 1121 may have a spiral configuration along at least a portion of sleeve 1120 placed in the stomach. Anti-kinking stripe may have a spiral configuration starting about 5 cm to about 15 cm from a proximal end of cuff 1110, and extending for about 15 cm to about 60 cm, such that the spiral extends from the esophagus through at least part of the gastric lumen, and in some extending through the gastric lumen into the duodenum. Anti-kinking stripe 1121 may have a spiral configuration with a pitch of about 20 mm. Anti-kinking stripe 1121 may have a straight or longitudinal configuration along at least a portion of sleeve 1120 placed in the intestine.

Sleeve 1120 may include one or more tails 1122. Tails 1122 may be folded to allow distal portion 1120d of sleeve 1120 to be sealed.

Sleeve 1120 may include one or more openings 1123. Openings 1123 may allow food to pass from an inside of sleeve 1120 to an outside of sleeve 1120. Openings 1123 may be formed in anti-kinking stripe 1121 and/or anywhere else in sleeve 1120. Openings 1123 may include one or more of holes, slits, channels, valves, flaps, and any other suitable openings.

Sleeve 1120 may include one or more sleeves described in U.S. Pat. Nos. 7,837,669, 7,881,797, 8,070,743, 8,118,774, 8,182,441, 8,182,459, 9,060,844, 9,675,489, and 9,757,264; and U.S. Patent Application Publication Nos. 2005/0177181, 2006/0155375, 2009/0012544, 2009/0012541, 2014/0188245, and 2016/0193063, which are incorporated by reference in their entireties.

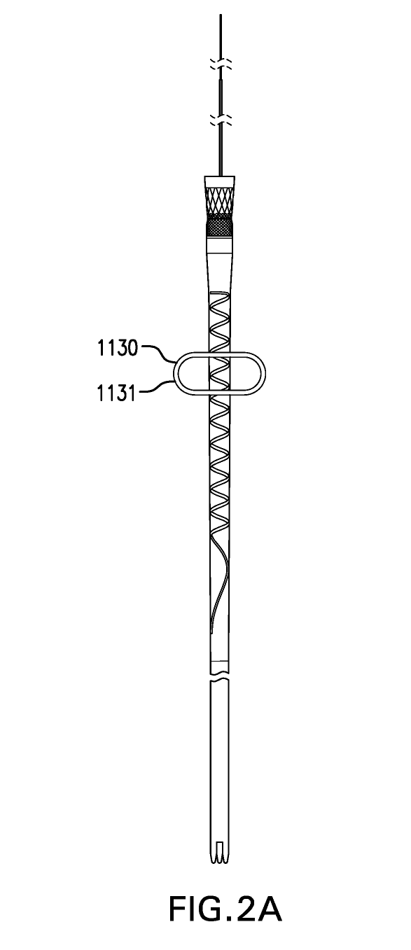

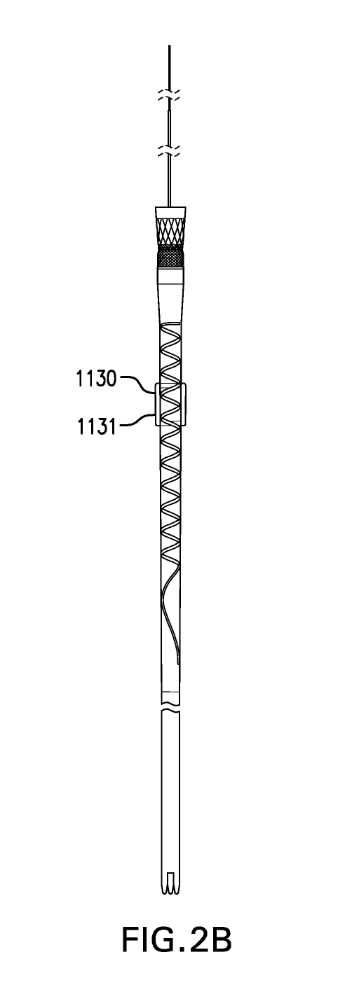

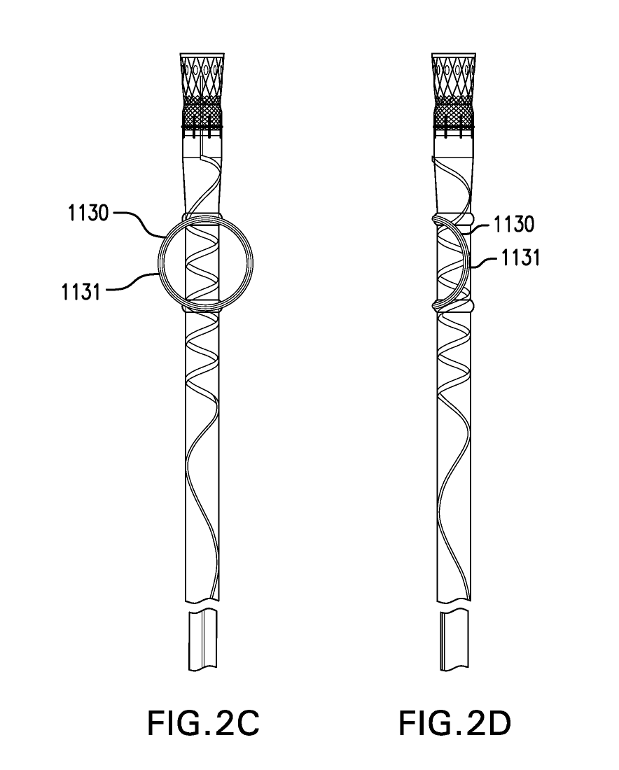

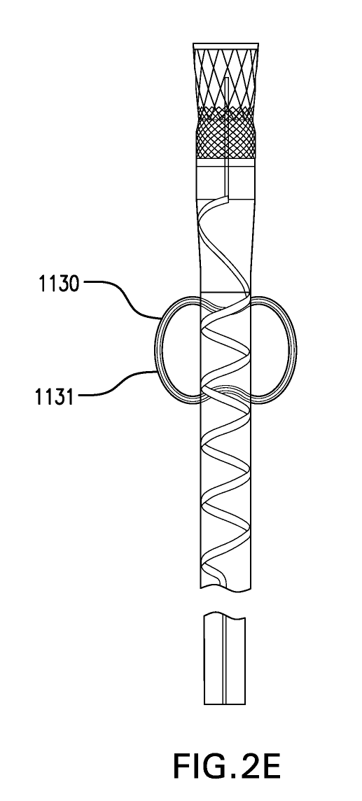

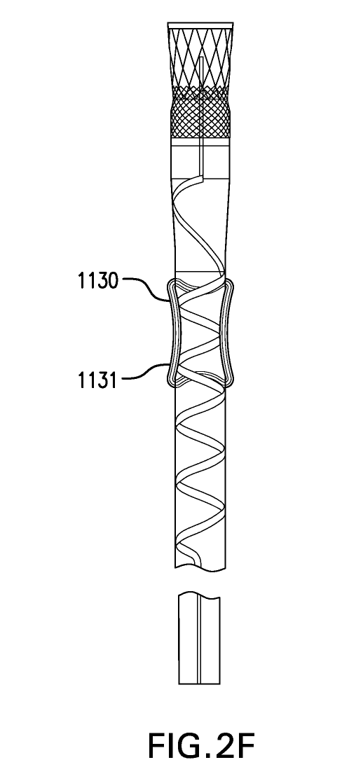

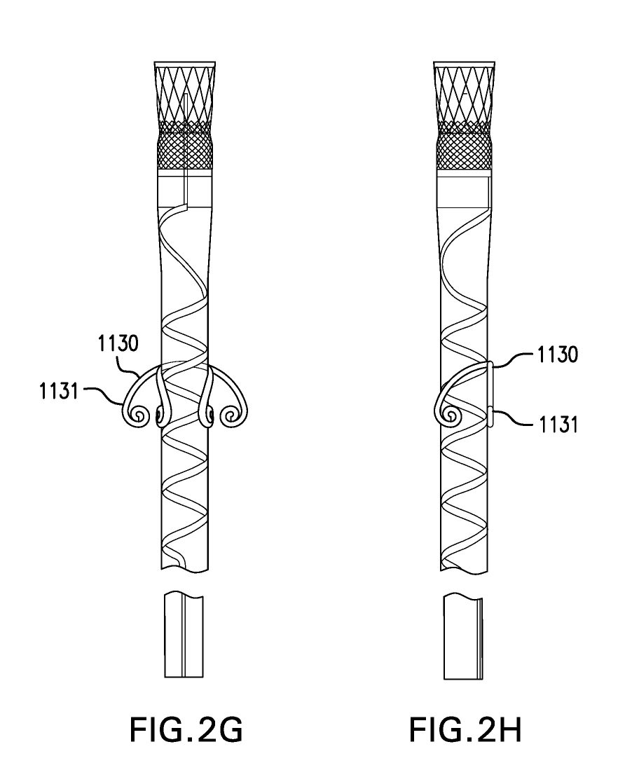

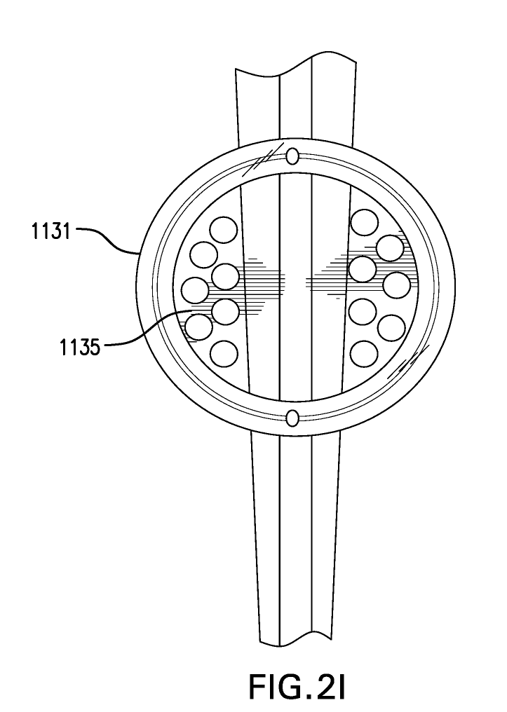

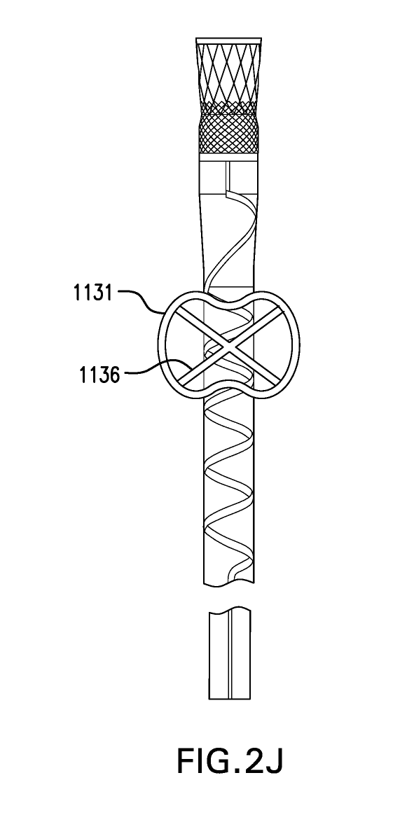

FIGS. 2A-2J, 3, and 4A-4D show various embodiments of a gastric retainer 1130. Cuff-sleeve assembly 1100 may include a gastric retainer 1130. Gastric retainer 1130 may be coupled to sleeve 1120. Gastric retainer 1130 may be coupled to anti-kinking stripe 1121.

Gastric retainer 1130 may be configured to be at least partially placed in one or more of the cardia, fundus, corpus, antrum, pylorus, and any other part of the stomach. Gastric retainer 1130 may limit travel of sleeve 1120 into the esophagus, such as during retching, belching, or vomiting. Gastric retainer 1130 may limit travel of sleeve 1120 into the intestine if cuff-sleeve assembly 1100 becomes detached from tether assembly 1300 and/or tether anchor 1400. The proximal opening of cuff 1110 and/or sleeve 1120 can be spaced axially distally apart from tether anchor 1400, such that cuff-sleeve assembly 1100 is entirely within the esophagus and does not traverse the fistula while tether anchor 1400 is anchored to another site. Tether anchor 1400 can be attached to a site other than, e.g., spaced apart and remote from a gastrointestinal tract structure including the skin or a bony structure for example. In some embodiments, the proximal opening of cuff 1110 and/or sleeve 1120 can be spaced distally apart by about or at least about 5 cm, 10 cm, 15 cm, 20 cm, 25 cm, or more, or ranges including any two of the aforementioned values.

Gastric retainer 1130 may have a collapsed configuration and a deployed configuration. Gastric retainer 1130 in the collapsed configuration may have a size that is small enough to pass through the esophagus for delivery and removal. Gastric retainer 1130 in the collapsed configuration may have a width of about 20 mm or less. Gastric retainer 1130 in the deployed configuration may have a size that is too large to pass through the esophagus and/or the intestine. Gastric retainer 1130 in the deployed configuration may have a width of about 30 mm to about 60 mm.

Gastric retainer 1130 may have an outer surface which is smooth. Gastric retainer 1130 may have an outer surface which is soft to reduce damage to tissue. Gastric retainer 1130 may have an outer surface with a hardness of about Shore 60A to about Shore 90A. Gastric retainer 1130 can be non-penetratingly deployed within, and movable with respect to the stomach, and not attached transmurally or partially transmurally through an esophageal, gastric, or GEJ wall for example.

Gastric retainer 1130 may have a stiffness sufficient to resist passing into the esophagus and/or intestine. Gastric retainer 1130 when oriented right side up may resist passing into the esophagus. Gastric retainer 1130 when oriented upside down may be more easily collapsed into an overtube or the esophagus.

FIGS. 2A-2J, in addition to FIGS. 1A-1B, show various embodiments of a gastric retainer 1130 including a ring 1131. Gastric retainer may include a ring 1131. Ring 1131 may be obround, circular, or any other suitable shape. Ring 1131 may be open or closed.

Ring 1131 may be in the deployed configuration when unconstrained. Ring 1131 may be constrained in the delivery configuration. Ring 1131 may be constrained by one or more of an overtube, a suture, loop snare, and any other suitable device. Ring 1131 may return to the deployed configuration when unconstrained. Ring 1131 in the deployed configuration may allow vomit to pass through and/or around ring 1131.

Ring 1131 may be made of one or more of a metal, plastic, or any other suitable material. Ring 1131 may be at least partially made of a shape memory metal such as nitinol. Ring 1131 may be at least partially made of a polymer, such as one or more of PEEK, polyurethane, nylon, polyester, PEBA, polyethylene, polypropylene, silicone, and any other suitable polymer. Ring 1131 may have a thickness of about 0.5 mm to about 5 mm.

In the embodiment shown in FIGS. 1A-1B, ring 1131 may be shaped like a circle with a tab 1134. Tab 1134 may facilitate removal of ring 1131 through an overtube or the esophagus.

FIGS. 2A-2B show deployed and collapsed configurations of another embodiment of a gastric retainer 1130 including a ring 1131. Ring 1131 may be shaped like an obround or a racetrack. The obround may have a shortest axis of about 20 cm to about 30 cm. The obround may have a longest axis of about 40 cm to about 50 cm. Ring 1131 may be pulled along the longest axis into an overtube or the esophagus for removal.

FIGS. 2C-2D show deployed and collapsed configurations of another embodiment of a gastric retainer 1130 including a ring 1131. Ring 1131 may be shaped like a circle.

FIGS. 2E-2F show deployed and collapsed configurations of another embodiment of a gastric retainer 1130 including a ring 1131. Ring 1131 may be shaped like an apple or a figure eight.

FIGS. 2G-2H show deployed and collapsed configurations of another embodiment of a gastric retainer 1130 including a ring 1131. Ring 1131 may be C-shaped or shaped like an arch.

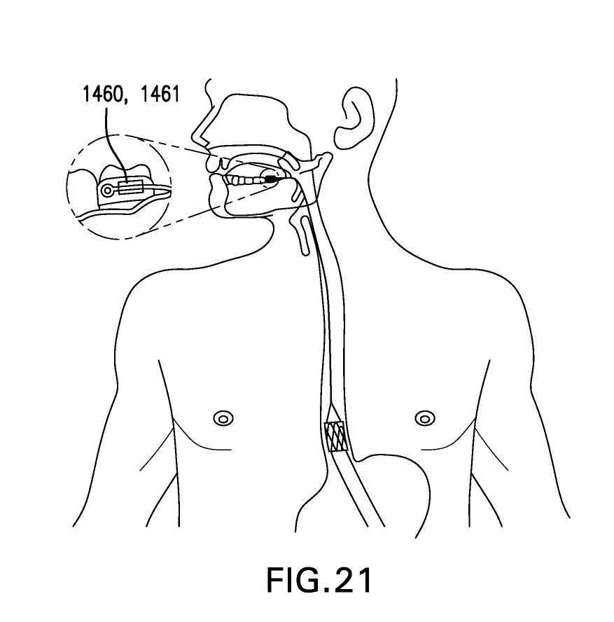

FIG. 21 shows one embodiment of a ring 1131 including a webbing 1135. Webbing 1135 may be made of a polymer or any other suitable material. Webbing 1135 may include one or more openings. FIG. 2J shows another embodiment of a ring 1131 including one or more struts 1136. Struts 1136 may be made of a same or similar material as core 1132 and/or cover 1133. Webbing 1135 and/or struts 1136 may provide additional stiffness to ring 1131. Webbing 1135 and/or struts 1136 may prevent sleeve 1120 becoming entangled with ring 1131.

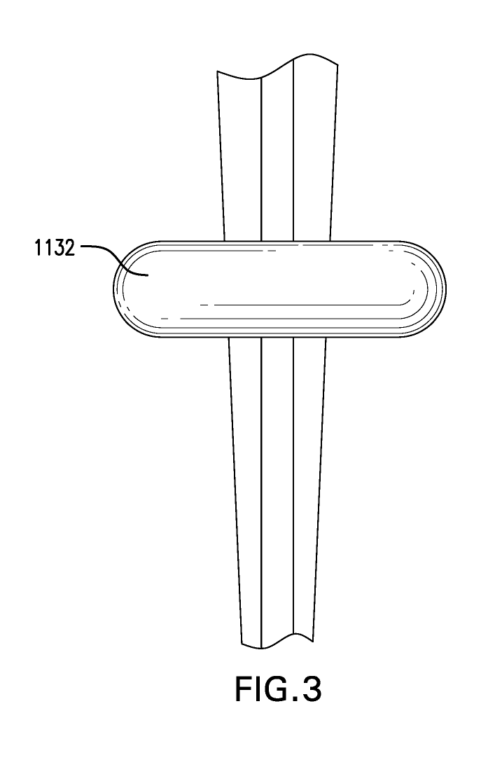

FIG. 3 shows one embodiment of a gastric retainer 1130 including a balloon 1132. Gastric retainer 1130 may include a balloon 1132. Balloon 1132 may be coupled to one side or encircle sleeve 1120. Balloon 1132 may be donut-shaped, bar-shaped, or any other suitable shape. Balloon 1132 may be deflated in the collapsed configuration. Balloon 1132 may be inflated with a gas and/or liquid into the deployed configuration.

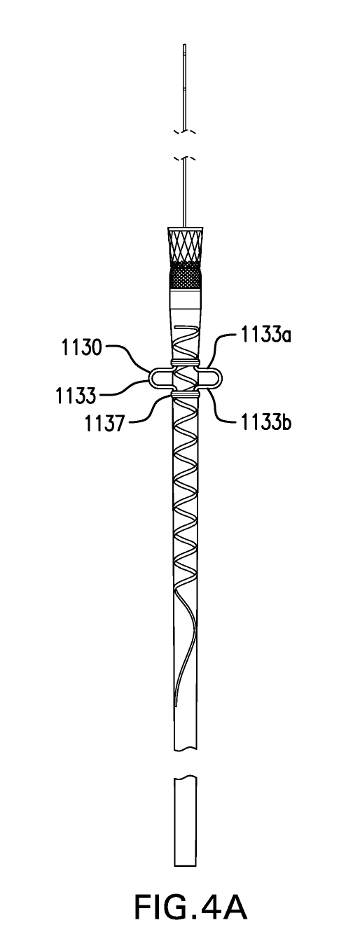

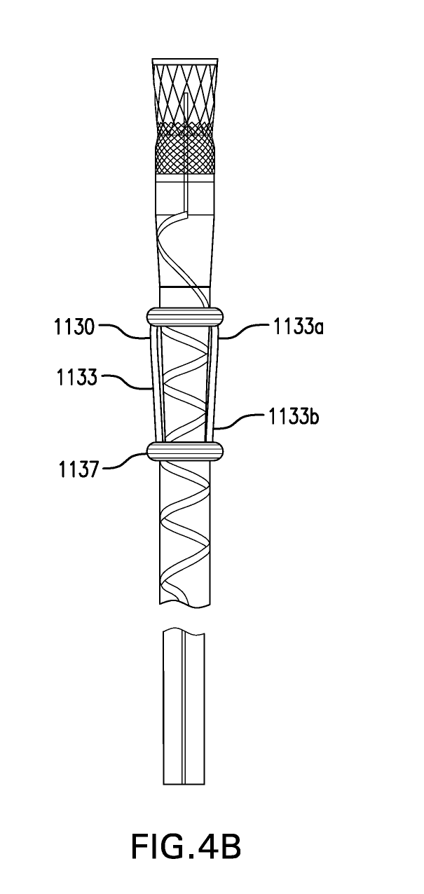

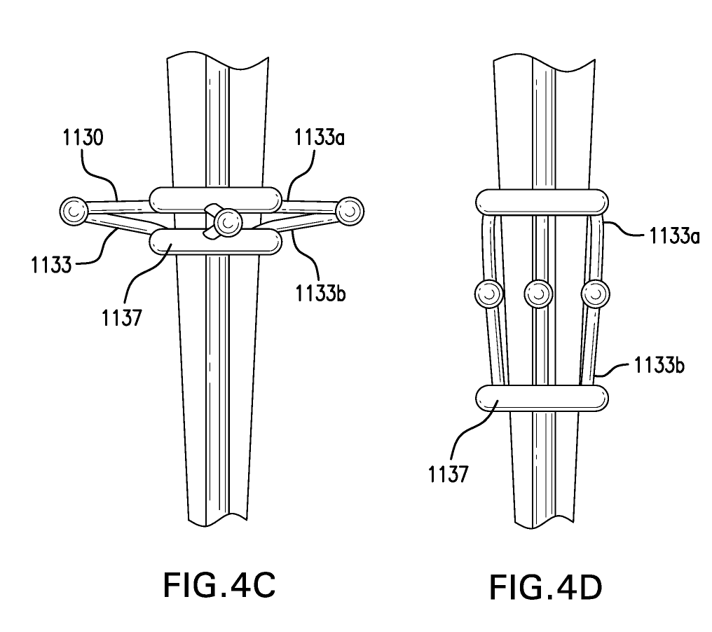

FIGS. 4A-4D show two embodiments of a gastric retainer 1130 including arms 1133. Gastric retainer 1130 may include one or more arms 1133.

Arms 1133 may be in the deployed configuration when unconstrained. Arms 1133 may be constrained in the delivery configuration. Arms 1133 may be constrained by one or more of an overtube, a suture, loop snare, and any other suitable device. Arms 1133 may return to the deployed configuration when unconstrained.

Arms 1133 in the collapsed configuration may lie against sleeve 1120. Arms 1133 in the deployed configuration may extend from sleeve 1120. Arms 1133 may be made of one or more of a metal, plastic, or any other suitable material. Arms 1133 may be at least partially made of a shape memory metal such as nitinol. Arms 1133 may be at least partially made of a polymer, such as one or more of PEEK, polyurethane, nylon, polyester, PEBA, polyethylene, polypropylene, silicone, and any other suitable polymer.

Each arm 1133 may have a first end 1133a coupled to sleeve 1120. Each arm 1133 may have a second end 1133b coupled to a slider 1137. Slider 1137 may be slidably coupled to sleeve 1120.

FIGS. 4A-4B show deployed and collapsed configurations of one embodiment of a gastric retainer 1130 with two arms 1133.

FIGS. 4C-4D show deployed and collapsed configurations of another embodiment of a gastric retainer 1130 with four arms 1133.

Gastrointestinal bypass device 1000 may include a tether assembly 1300. Tether assembly 1300 may maintain cuff-sleeve assembly 1100 in a particular portion of the gastrointestinal tract.

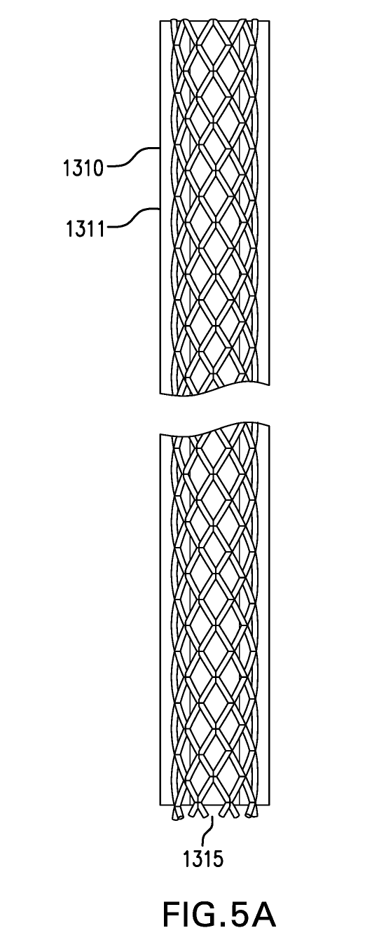

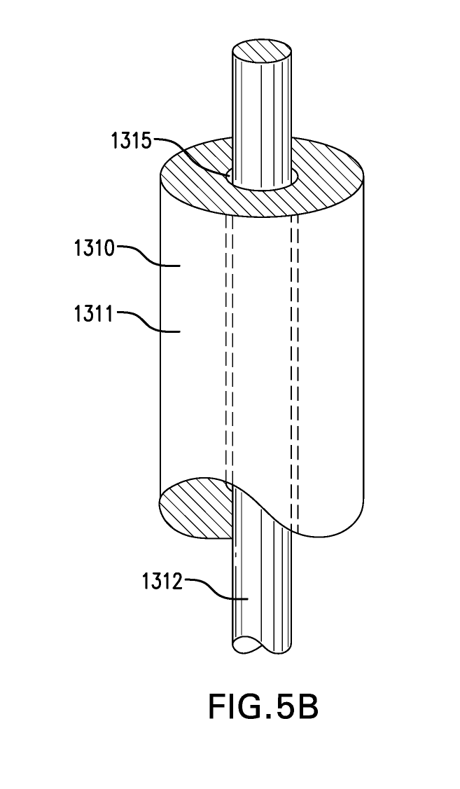

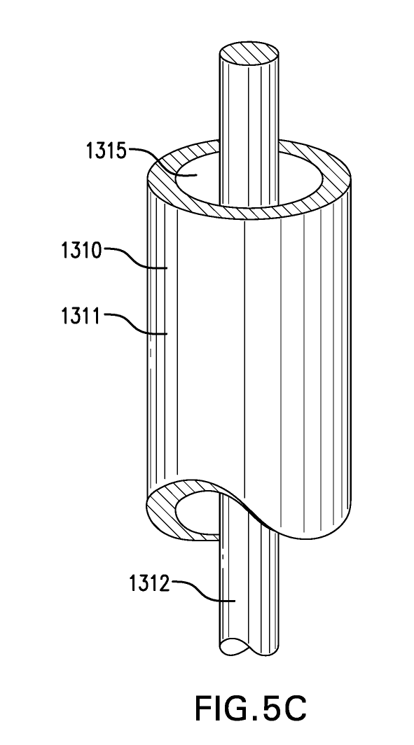

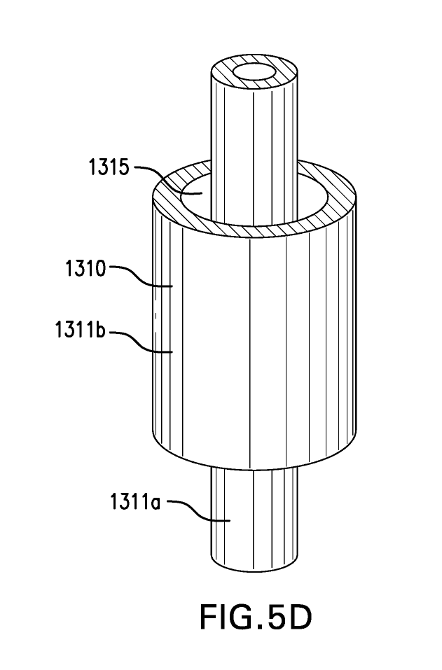

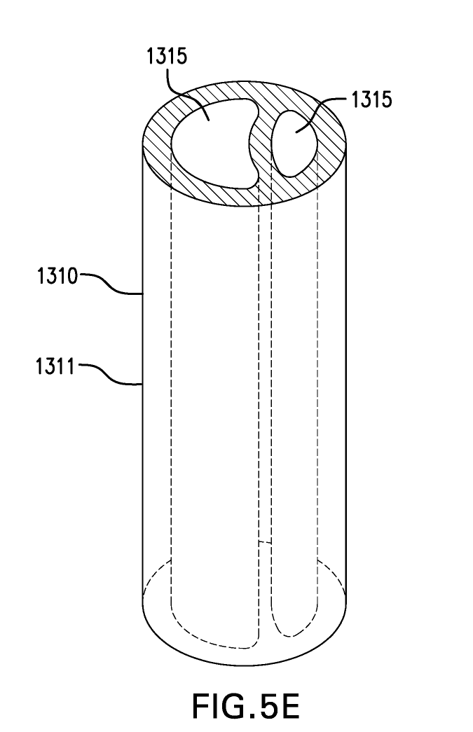

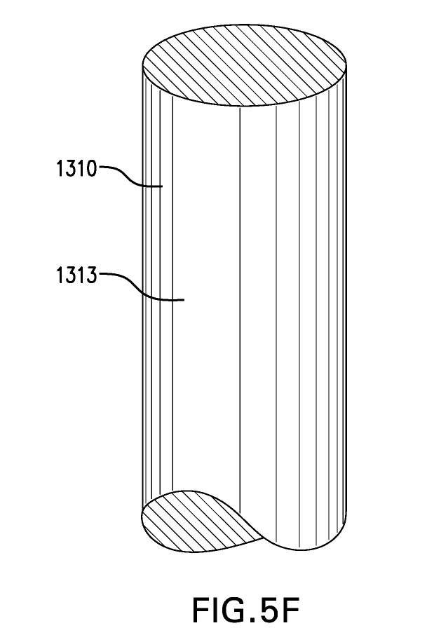

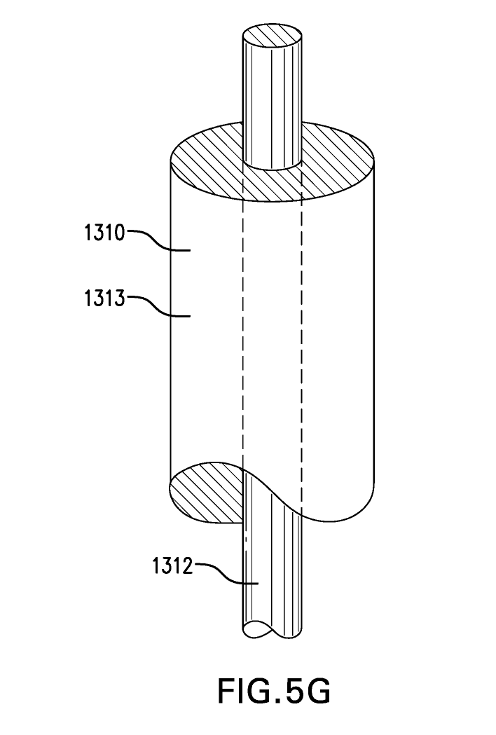

FIGS. 5A-5H show various embodiments of a tether 1310. Tether assembly 1300 may include a tether 1310. Tether 1310 may couple cuff-sleeve assembly 1100 to an anchor site. Tether 1310 may be configured to be placed at least partially in the esophagus. Tether 1310 may be configured to be placed through a fistula between the esophagus and the skin.

Tether 1310 may include a proximal portion 1310p and a distal portion 1310d. Proximal portion 1310d of tether 1310 may be configured to be coupled to an anchor site. Distal portion 1310d of tether 1310 may be coupled to one or more of cuff 1110, sleeve 1120, and gastric retainer 1130. Distal portion 1310d of tether 1310 may extend along at least a portion of sleeve 1120 placed in the stomach, and may reduce kinking and/or twisting of sleeve 1120.

Tether 1310 may have a length sufficient to extend from cuff-sleeve assembly 1100 in an appropriate direction, such as proximally from cuff 1110 and/or sleeve 1120 up the esophagus to an anchor site (e.g., via an esophageal fistula in some cases), such as the skin of the neck or upper chest, for example. Tether 1310 may have a length to cuff 1110 of about 10 cm to about 30 cm. Tether 1310 may elongate little to none under tension. Tether 1310 may elongate less than 10% under tension. Tether 1310 may elongate less than 10% when subject to tension less than 20 N. Tether 1310 may be cut to a particular length. Tether 1310 may be removably coupled to different locations on one or more of cuff 1110, sleeve 1120, and gastric retainer 1130, effectively adjusting the length of tether 1310.

Tether 1310 may have a thickness that is uniform or varying along its length. Tether 1310 may have a thickness of about 5 Fr to about 16 Fr (about 1.67 mm to about 5.33 mm). Tether 1310 may have a thickness which distributes forces over an area of tissue sufficient to reduce erosion and/or ulceration of the inside of the esophageal wall where tether 1310 passes through the esophageal wall. Tether 1310 may have a thickness of at least about 8 Fr (about 2.67 mm) where tether 1310 passes through the esophageal wall.

Tether 1310 may have a tensile strength sufficient to withstand pulling forces exerted on cuff-sleeve assembly 1100, such as from peristaltic movement. Tether 1310 may have a stiffness which distributes pulling forces over an area of tissue sufficient to reduce erosion and/or ulceration of the inside of the esophageal wall where tether 1310 passes through the esophageal wall. Tether 1310 may have a bending stiffness which holds or carries tether 1310 away from the inside of the esophageal wall where tether 1310 passes through the esophageal wall. Tether 1310 may have a bending stiffness which does not allow tether 1310 to rest against the inside of the esophageal wall immediately below where tether 1310 passes through the esophageal wall. Tether 1310 may have a bending stiffness of about 12 N·mm² to about 50 N·mm² where tether 1310 passes through the esophageal wall, as measured by a three-point bending test with supports 15 mm apart and a deflection of 3 mm.

Tether 1310 may have an outer surface which is smooth. Tether 1310 may have an outer surface which is soft to reduce damage to tissue. Tether 1310 may have an outer surface with a hardness of about Shore 70A to about Shore 100A.

Proximal portion 1310p of tether 1310 may be tapered and/or rounded. The taper can be configured such that the tether 1310 increases in thickness from proximal portion 1310p to a more distal zone to facilitate a small initial puncture, allowing tether 1310 to dilate the fistula during placement. Tether 1310 may include an antimicrobial coating.

Tether 1310 may include a tube 1311. Tube 1311 may be at least partially made of a polymer, such as one or more of polyurethane, nylon, polyester, PEBA, polyethylene, polypropylene, silicone, and any other suitable polymer. Tube 1311 may be reinforced with one or more fibers and/or wires. Fibers may include one or more of nylon, polyester, aramid such as KEVLAR®, and other suitable fibers. Wires may include stainless steel and/or any other suitable wires. The fibers and/or wires may be woven and/or braided.

Tube 1311 may include at least one lumen 1315. Lumen 1315 may be of any suitable size and shape. Lumen 1315 may allow a fluid to be delivered and/or stored. Lumen 1315 may allow for fluids and/or materials to be aspirated or removed. Lumen 1315 may allow tools and instruments to be passed through and procedures to be performed. Lumen 1315 may allow electrical leads to be placed through to reach sensors on one or more of tether 1310, cuff 1110, and sleeve 1120.

Tube 1311 may include one or more side openings 1316. Side openings 1316 may be in fluid communication with lumen 1315. Side openings 1316 may be formed at distal portion 1310d of tether 1310, or anywhere along a length of tether 1310.

Side openings 1316 may allow a fluid to be delivered. Side openings 1316 may have a size of about 0.05 mm or less, which may allow fluids to diffuse out passively over time. Side openings 1316 may have a size of more than 0.05 mm, which may allow fluids to flow out freely.

Tether 1310 may include a core 1312. Core 1312 may include a suture. A suture may be plain and/or barbed. Core 1312 may include a wire. A wire may be made of stainless steel wire and/or any other suitable material. Core 1312 may have a thickness of about 0.3 mm to about 0.8 mm.

Tether 1310 may include a rod 1313. Rod 1313 may be at least partially made of a polymer, such as one or more of polyurethane, nylon, polyester, PEBA, polyethylene, polypropylene, silicone, and any other suitable polymer. Rod 1313 may be reinforced with one or more fibers and/or wires, such as a mesh and/or a braid.

FIG. 5A shows one embodiment of a tether 1310 including a tube 1311. Tube 1311 may include a lumen 1315. Tube 1311 may be reinforced with a mesh and/or a braid. Tube 1311 may be formed on the inside, outside, or around the mesh and/or braid.

FIGS. 5B-5C show two embodiments of a tether 1310 including a tube 1311 and a core 1312. Core 1312 may be at least partially placed within lumen 1315 of tube 1311.

FIG. 5D shows one embodiment of a tether 1310 including an inner tube 1311a and an outer tube 1311b. Inner tube 1311a may be at least partially placed within lumen 1315 of outer tube 1311b.

FIG. 5E shows one embodiment of a tether 1310 including a tube 1311. Tube 1311 may include two lumens 1315.

FIG. 5F shows one embodiment of a tether 1310 including a rod 1313.

FIG. 5G shows one embodiment of a tether 1310 including a rod 1313 and a core 1312. Rod 1313 may be extruded over core 1312. At least a portion of rod 1313 may be removable from core 1312.

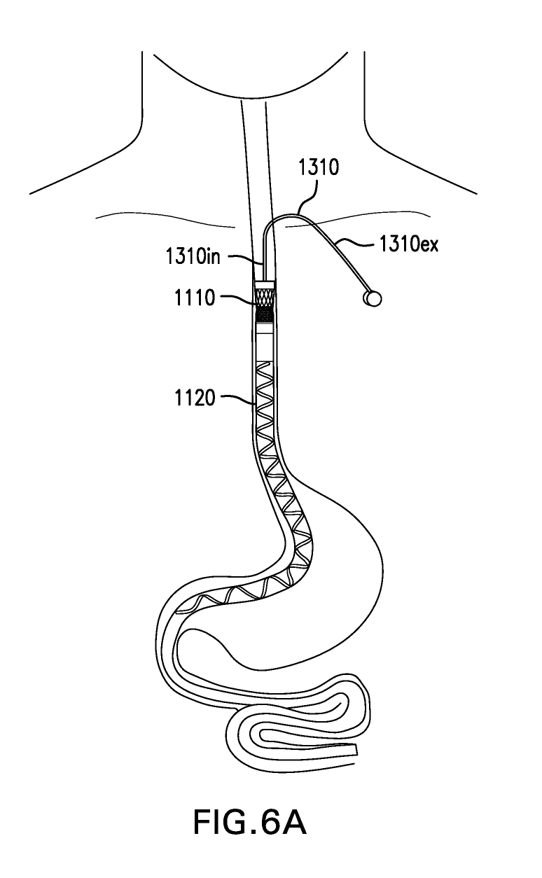

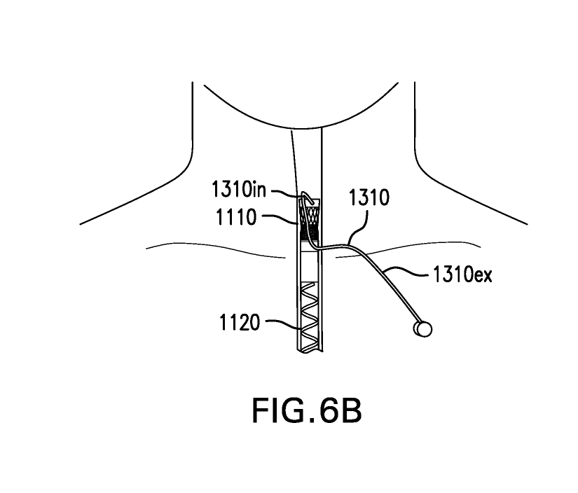

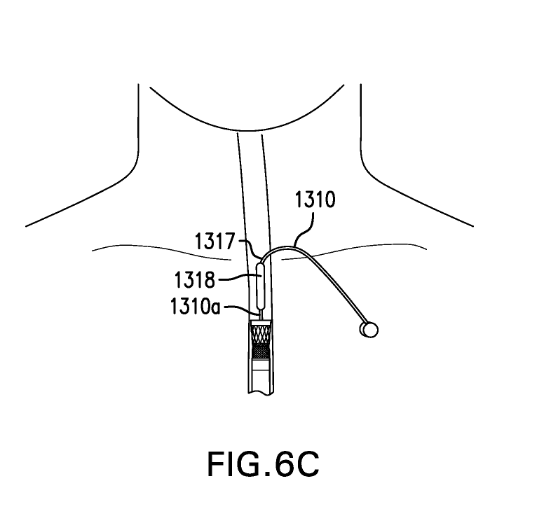

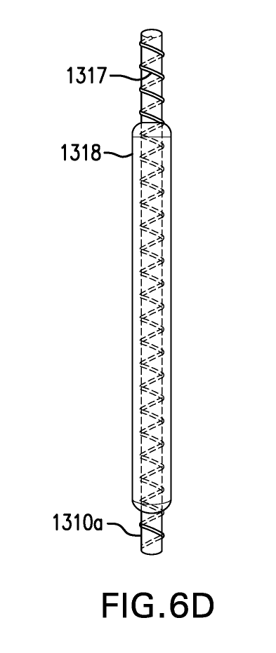

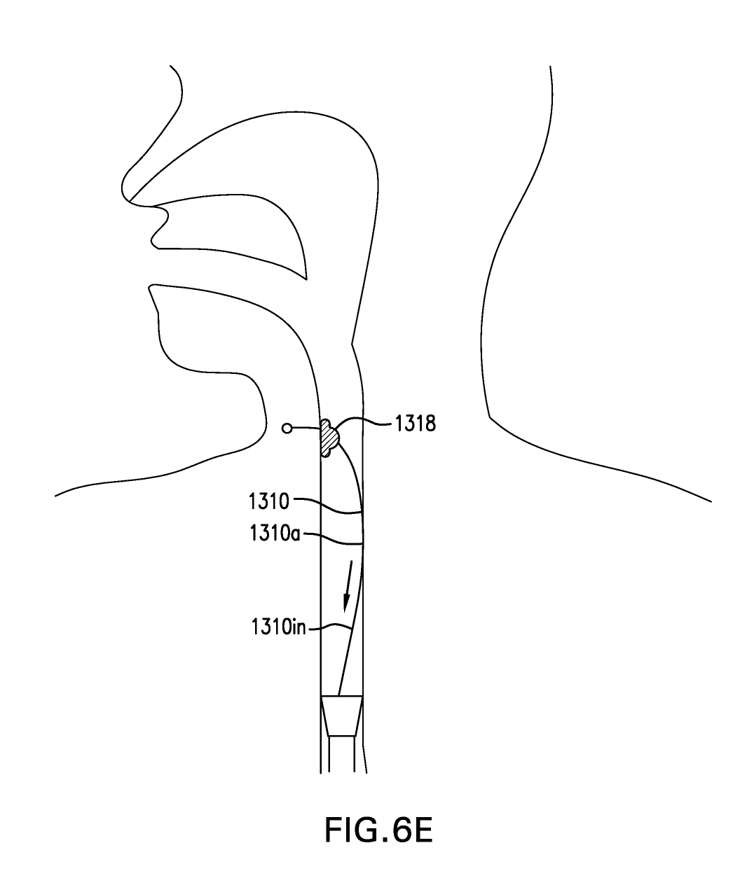

FIGS. 6A-6B show a tether 1310 with intraesophageal portion 1310in extended distally and proximally, respectively. Tether 1310 may include an intraesophageal portion 1310in and an extraesophageal portion 1310ex. Intraesophageal portion 1310in may have a length which limits proximal travel of cuff 1110 and/or sleeve 1120, such as during retching, belching, or vomiting. Intraesophageal portion 1310in may have a length which prevents cuff 1110 and/or sleeve 1120 from traveling into the throat. Intraesophageal portion 1310in may have a length of about 3 cm to about 12 cm.

FIGS. 6C-6D show one embodiment of a tether 1310 including a stiffened portion 1310a. Tether 1310 may include a stiffened portion 1310a. Stiffened portion 1310a may include at least a portion of intraesophageal portion 1310in and/or distal portion 1310d of tether 1310. Stiffened portion 1310a may have a stiffness which limits proximal travel of cuff 1110 and/or sleeve 1120, such as during retching, belching, or vomiting. Stiffened portion 1310a may have a stiffness which prevents cuff 1110 and/or sleeve 1120 from traveling proximal to where tether 1310 passes through the esophageal wall, or into the throat. Stiffened portion 1310a may be configured to bend no more than about 180 degrees under compression. At least a portion of stiffened portion 1310a may be made of a material with increased stiffness.

Stiffened portion 1310a may include a stiffening element 1317. Stiffening element 1317 may include a coil. Stiffening element 1317 may be made of one or more of nitinol, spring wire, and any other suitable material. Stiffening element 1317 may be wrapped around tether 1310 or at least partially embedded in tether 1310.

Tether 1310 may include a stop 1318. Stop 1318 may be coupled to intraesophageal portion 1310in. Stop 1318 may limit proximal travel of cuff 1110 and/or sleeve 1120 by preventing intraesophageal portion 1310in of tether 1310 from being pushed into the fistula. Stop 1318 may include a bumper or tube with rounded ends. Stop 1318 may have a thickness greater than that of tether 1310. Stop 1318 may have a thickness of about 10 Fr to about 16 Fr (about 3.33 mm to about 5.33 mm). Stop 1318 may also increase the stiffness of interesophageal portion 1310in.

FIG. 6E shows another embodiment of a tether 1310 including a stop 1318. Stop 1318 may include a flange. Stop 1318 may be placed against an inside of the esophageal wall.

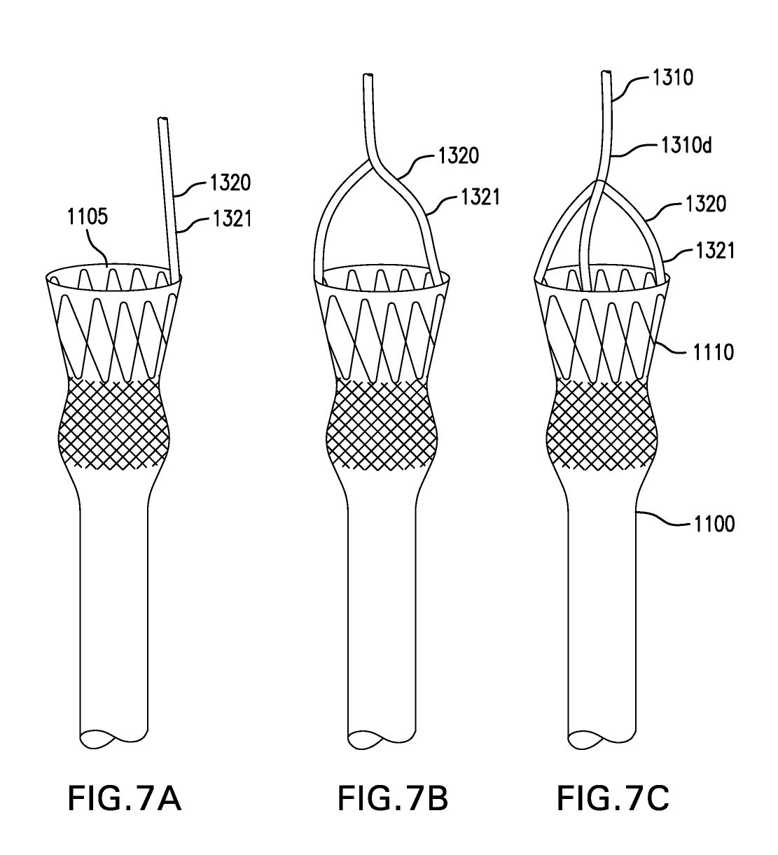

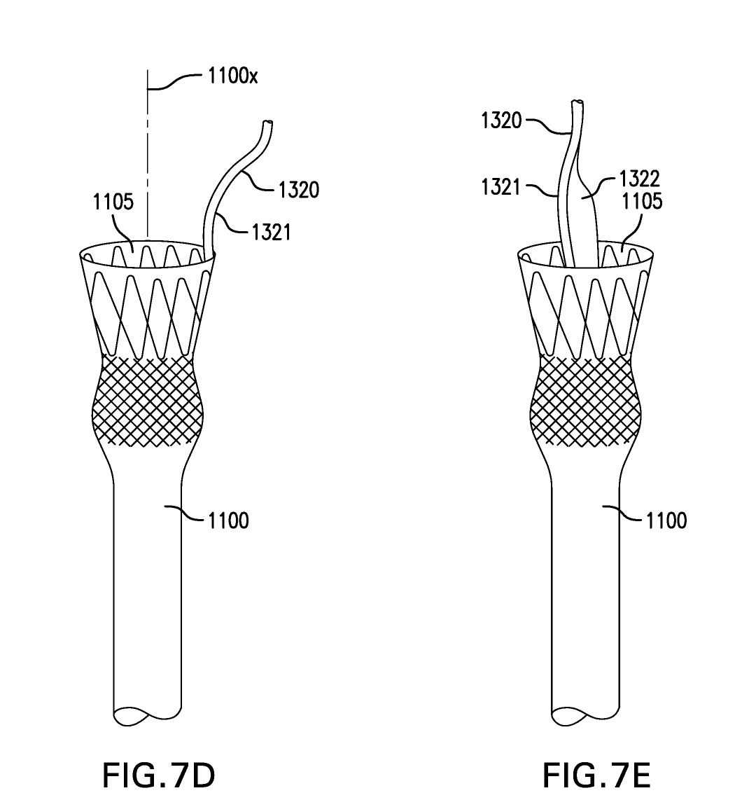

FIGS. 7A-7E show various embodiments of a bridle 1320. Tether assembly 1300 may include a bridle 1320. Bridle 1320 may couple tether 1310 to cuff-sleeve assembly 1100. Bridle 1320 may be coupled to distal portion 1310d of tether 1310. Bridle 1320 may be coupled to cuff-sleeve assembly 1100. Bridle 1320 may direct tether 1310 away from longitudinal axis 1100x of cuff-sleeve assembly 1100 and closer to the esophageal wall, to reduce the likelihood of tether 1310 retaining or obstructing food and other items passing into cuff 1110. Bridle 1320 may help maintain the orientation of cuff 1110 relative to the longitudinal axis of the esophageal lumen and reduce the likelihood of cuff 1110 tipping. Bridle 1320 may be positioned to provide unobstructed access from lumen 1315 of tether 1310 to lumen 1105 of cuff-sleeve assembly 1100 and/or an outside of cuff-sleeve assembly 1100.

Bridle 1320 may include one or more risers 1321. Risers 1321 may be made of one or more of the same or similar materials as tether 1310. At least one riser 1321 may include a continuation or extension of tether 1310.

FIGS. 7A-7C show embodiments of a bridle 1320 including a single riser 1321, two risers 1321, and three risers 1321, respectively. Risers 1321 may be coupled to cuff-sleeve assembly 1100 evenly or irregularly around cuff-sleeve assembly 1100.

FIG. 7D shows another embodiment of a bridle 1320 including at least one riser 1321 with increased stiffness. Riser 1321 may have a stiffness sufficient to prevent tether 1310 and/or risers 1321 from sagging into and/or obstructing lumen 1105 of cuff-sleeve assembly 1100. At least a portion of riser 1321 may be made of a material with increased stiffness. Riser 1321 may include a stiffening element, such as a wire. Riser 1321 may be curved or oriented away from longitudinal axis 1100x of cuff-sleeve assembly 1100.

FIG. 7E shows another embodiment of a bridle 1320 including at least one riser 1321 with a fin 1322. Riser 1321 may include a fin 1322 to prevent tether 1310 and/or risers 1321 from sagging into and/or obstructing lumen 1105 of cuff-sleeve assembly 1100. Fin 1322 may be coupled along at least part of a length of riser 1321. Fin 1322 may have a stiffness sufficient to support riser 1321. Fin 1322 may be flexible to allow cuff-sleeve assembly 1100 to compress. Fin 1322 may allow food and other items to pass on either side of fin 1322.

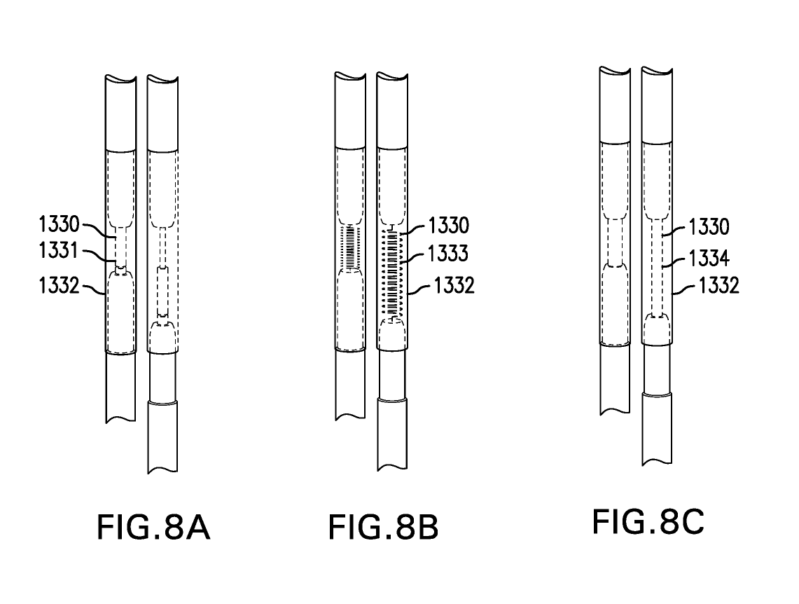

FIGS. 8A-8C show various embodiments of an elongation element 1330. Tether assembly 1300 may include an elongation element 1330. Elongation element 1330 may be coupled anywhere along tether 1310 and/or bridle 1320. Elongation element 1330 may be coupled at or near distal portion 1310d of tether 1310. Elongation element 1330 may be coupled between tether 1310 and bridle 1320. Elongation element 1330 may lengthen by a limited amount, and then return to an original length. Elongation element 1330 may lengthen by about 1 cm to about 3 cm. Elongation element 1330 may serve as a shock absorber and dampen movement of cuff-sleeve assembly 1100, such as with swallowing and changes in posture. Elongation element 1330 may reduce tugging sensations and/or tissue damage caused by movement of cuff-sleeve assembly 1100.

FIG. 8A shows one embodiment of an elongation element 1330. Elongation element 1330 may include a hydraulic or gas-filled cylinder 1331. Hydraulic or gas-filled cylinder 1331 may be similar to an automobile shock absorber or gas strut. Elongation element 1330 may include a cover 1332.

FIG. 8B shows another embodiment of an elongation element 1330. Elongation element 1330 may include a spring 1333. Spring 1333 may be made of one or more of a metal, plastic, and any other suitable material.

FIG. 8C shows another embodiment of an elongation element 1330. Elongation element 1330 may include an elastic segment 1334 at least partially made of an elastic polymer. Elongation element 1330 may include an elastic segment 1334 at least partially made of silicone.

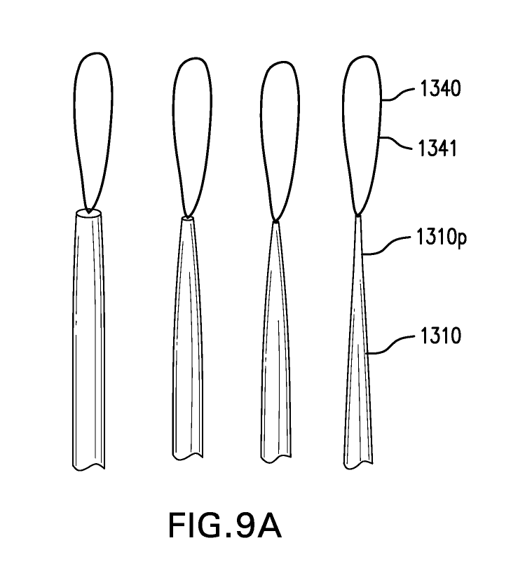

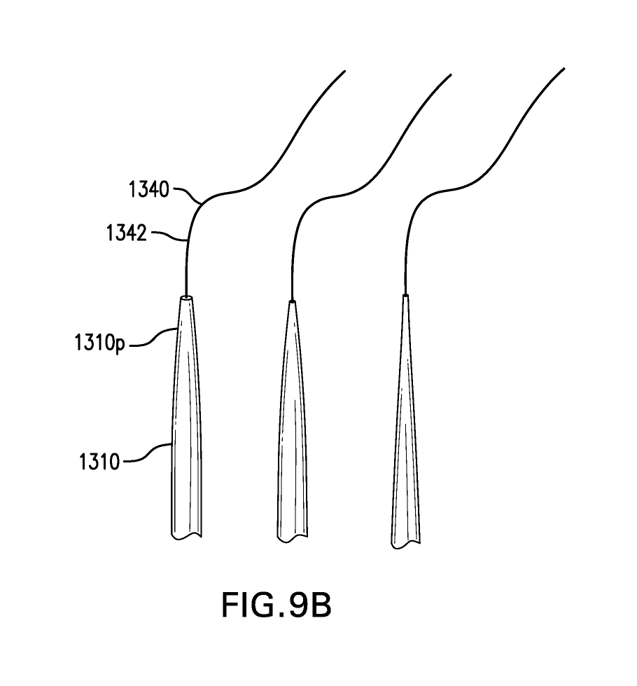

FIGS. 9A-9B show two embodiments of a pull tab 1340. Tether assembly 1300 may include a pull tab 1340. Pull tab 1340 may be coupled to proximal portion 1310p of tether 1310. Pull tab 1340 may be coupled to tether 1310 with one or more of stitches, an adhesive, and any other suitable device or method. Pull tab 1340 may provide a way to manipulate proximal portion 1310p of tether 1310. Pull tab 1340 may provide a way to attach other devices to proximal portion 1310p of tether 1310. Pull tab 1340 may include a suture and/or a wire. Pull tab 1340 may include a portion of core 1312.

FIG. 9A shows one embodiment of pull tab 1340. Pull tab 1340 may include a loop 1341.

FIG. 9B shows another embodiment of pull tab 1340. Pull tab 1340 may include an unlooped element 1342.

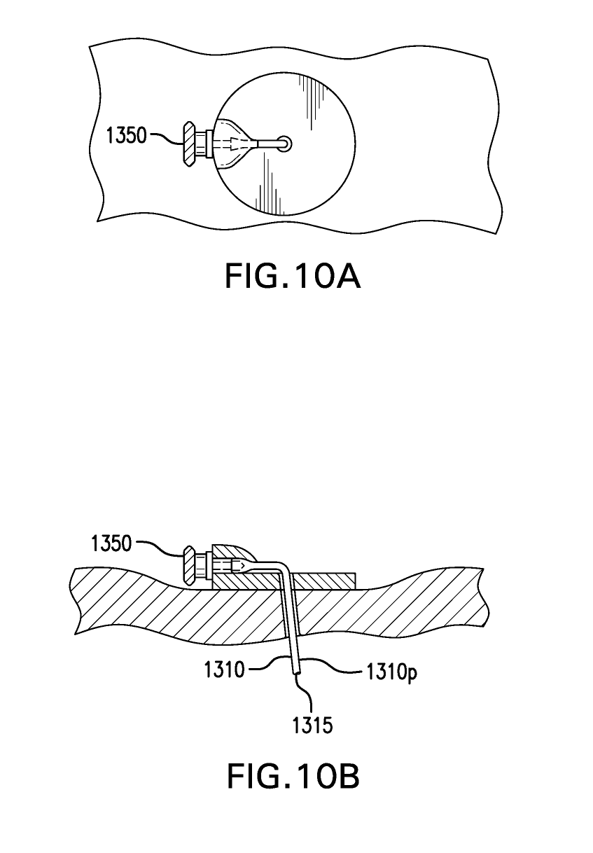

FIGS. 10A-10B show one embodiment of an access port 1350. Tether assembly 1300 may include an access port 1350. Access port 1350 may be coupled to proximal portion 1310p of tether 1310. Access port 1350 may provide access to one or more lumens 1315 of tether 1310.

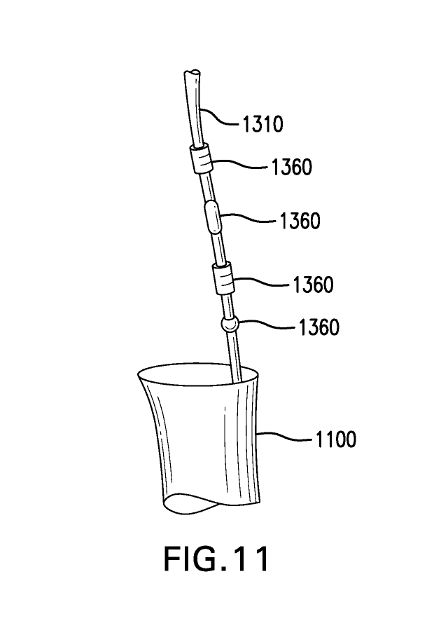

FIG. 11 shows one embodiment of sensors 1360. Tether assembly 1300 may include one or more sensors 1360. Sensors 1360 may be placed anywhere along tether 1310 and/or bridle 1320. Sensors 1360 may be connected to electrical leads passed through a lumen 1315 of tether 1310. Sensors 1360 may include one or more of temperature, pressure, pH, impedance, heart rate, accelerometry, electrocardiography, respiration, and any other suitable sensor.

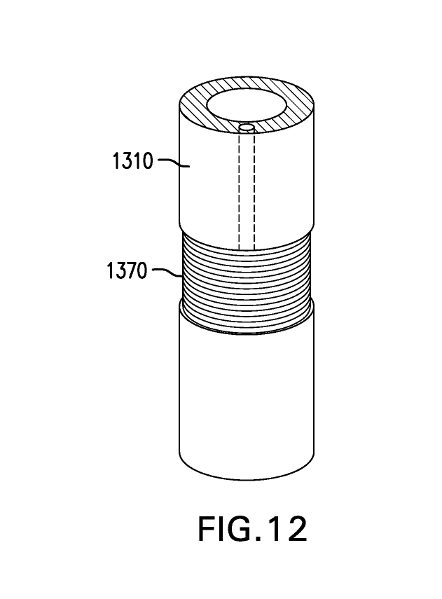

FIG. 12 shows one embodiment of electrodes 1370. Tether assembly 1300 may include one or more electrodes 1370. Electrodes 1370 may be placed anywhere along tether 1310 and/or bridle 1320. Electrodes 1370 may be connected to electrical leads passed through a lumen 1315 of tether 1310. Electrodes 1370 may deliver electrical pulses. Electrodes 1370 may be used to deliver stimulating pulses in the immediate vicinity of the lower esophageal sphincter to increase sphincter tone for prevention or amelioration of gastroesophageal reflux. Electrodes 1370 may be used to deliver mucosal stimulation pulses to induce gastric distension and suppress gastric motility in a way that may increase postprandial satiation. Electrodes 1370 may be used in a closed-loop fashion with sensors 1360 and other sensors.

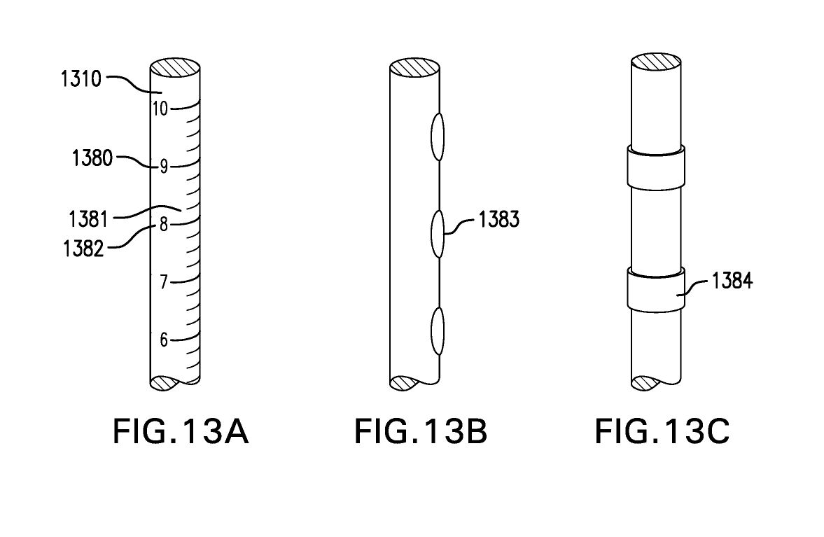

FIG. 13A-13C show various embodiments of markings 1380. Tether assembly 1300 may include one or more markings 1380. Markings 1380 may be formed on tether 1310 and/or bridle 1320. Markings 1380 may be radiopaque. Markings 1380 may be used to guide placement of cuff-sleeve assembly 1100. Markings 1380 may include one or more of lines, numbers, dots, bands, and any other suitable markings.

FIG. 13A shows one embodiment of markings 1380. Markings 1380 may include graduated lines 1381. Markings 1380 may include one or more numbers 1382 indicating a distance from cuff-sleeve assembly 1100.

FIG. 13B shows another embodiment of markings 1380. Markings 1380 may include one or more dots 1383.

FIG. 13C shows another embodiment of markings 1380. Markings 1380 may include one or more bands 1384.

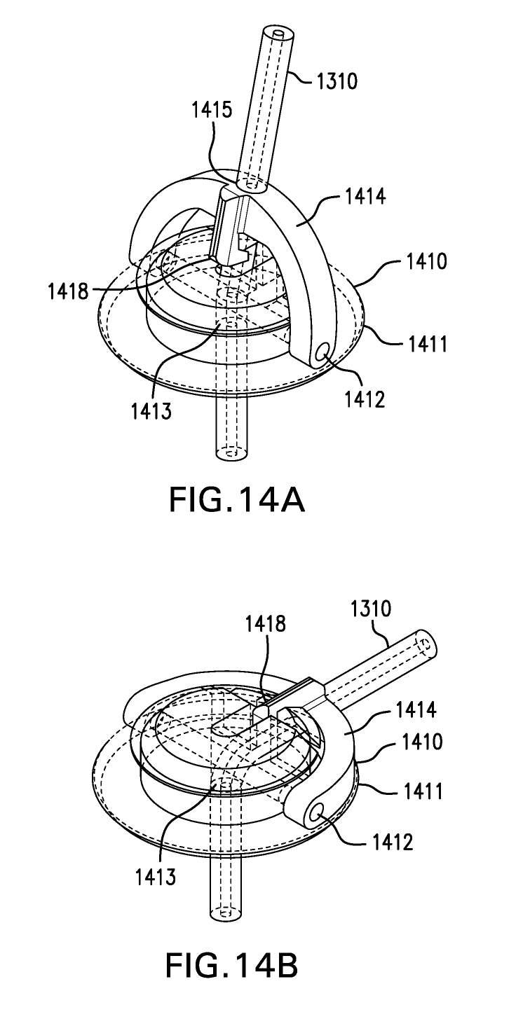

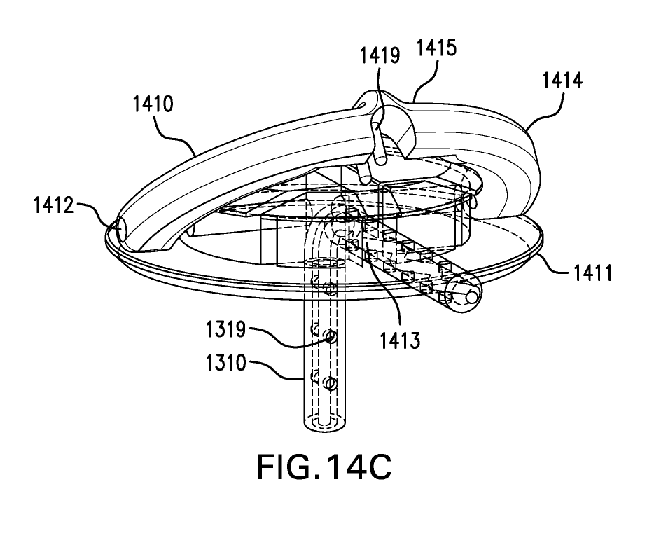

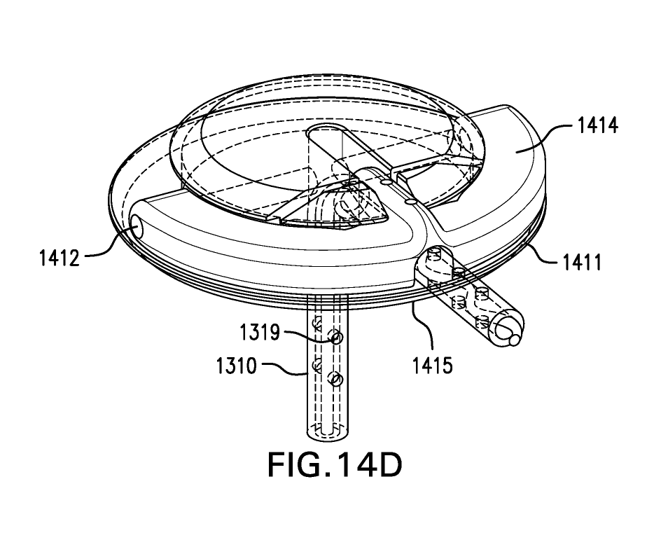

FIGS. 14A-14D, 15A-15B, 16A-16B, 17A-17H, 18A-18B, 19A-19B, 20-21, and 22A-22B show various embodiments of a tether anchor 1400. Tether anchor 1400 may be coupled to tether 1310. Tether anchor 1400 may secure tether 1310 to an anchor site.

Tether anchor 1400 may be coupled to a tether 1310 which is placed through a fistula between the esophagus and the skin (i.e., an esophago-cutaneous fistula). Tether anchor 1400 may be placed on the skin and/or under the skin. Tether anchor 1400 may be placed in the fistula. Tether anchor 1400 may be coupled to a bone.

Tether anchor 1400 may be coupled to a tether 1310 which is not placed through a fistula between the esophagus and the skin. Tether anchor 1400 may be coupled to a tooth, such as a molar. Tether anchor 1400 may be coupled to the nose, such as the nasal septum and/or sides of the nose. Tether anchor 1400 may be coupled to the hard and/or soft palate.

Tether anchor 1400 coupled to one anchor site may be used in combination with at least one other tether anchor 1400 coupled to another anchor site.

FIGS. 14A-14D show two embodiments of a tether anchor 1400 including a skin button 1410. Skin button 1410 may be configured to be placed on the skin. Skin button 1410 may be placed on the skin at the fistula. Skin button 1410 may be placed on the skin away from the fistula, which may reduce the likelihood of infection. Skin button 1410 may be placed on the skin about 8 cm away from the fistula. Tether 1310 may be tunneled under the skin from the fistula to skin button 1410.

Skin button 1410 may have an unlocked configuration and a locked configuration. Skin button 1410 in the unlocked configuration may allow tether 1310 to be moved in and out of the fistula and adjusted. Skin button 1410 in the locked configuration may prevent tether 1310 from moving. Skin button 1410 may accommodate access port 1350.

Skin button 1410 may include a base plate 1411. Base plate 1411 may have a bottom surface configured to be placed on the skin. Base plate 1411 may have a bottom surface with a soft material, such as foam for example. Base plate 1411 may have an antimicrobial treatment. Base plate 1411 may be large enough to comfortably distribute forces from tether 1310. Base plate 1411 may include a hinge 1412. Base plate 1411 may include a channel 1413 through which tether 1310 may be passed.

Skin button 1410 may include a latch 1414. Latch 1414 may be coupled to base plate 1411 by hinge 1412. Latch 1414 may include a channel 1415 through which tether 1310 may be passed. Channel 1415 of latch 1414 may be aligned with channel 1413 of base plate 1411.

FIGS. 14A-14B show unlocked and locked configurations of one embodiment of a tether anchor 1400 including a skin button 1410. Skin button 1410 in the locked configuration may pinch or squeeze tether 1310 between latch 1414 and base plate 1411. Skin button 1410 may include one or more pinchers 1418 which protrude from latch 1414 and/or base plate 1411 to pinch or squeeze tether 1310 more tightly. Pinchers 1418 may be at least partially in and/or aligned with channel 1415 and/or channel 1413.

FIGS. 14C-14D show unlocked and locked configurations of another embodiment of a tether anchor 1400 including a skin button 1410. Skin button 1410 may include one or more pins 1419 which protrude from latch 1414 and/or base plate 1411. Pins 1419 may be configured to mate with one or more holes 1319 formed in tether 1310. Skin button 1410 in the locked configuration may hold tether 1310 with pins 1419. Pins 1419 may be at least partially in and/or aligned with channel 1415 and/or channel 1413.

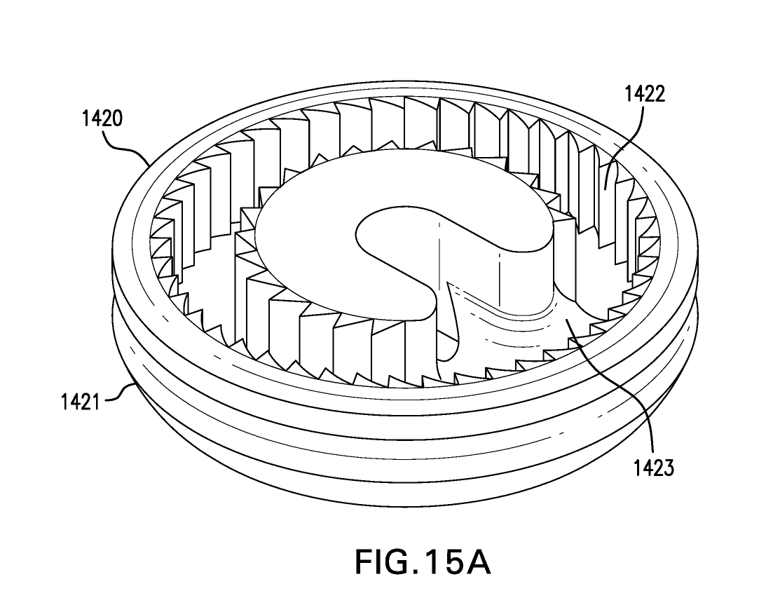

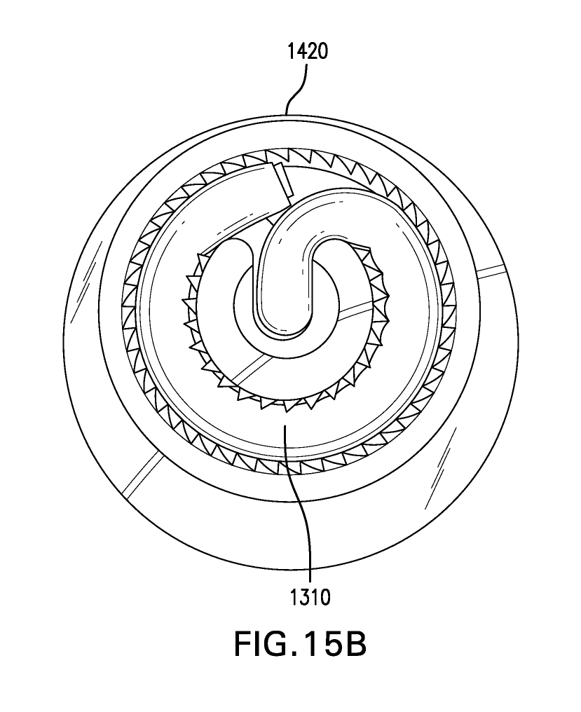

FIGS. 15A-15B show unsecured and secured configurations of another embodiment of a tether anchor 1400 including a skin button 1420. Skin button 1420 may be configured to be placed on the skin. Skin button 1420 may be placed on the skin at the fistula. Skin button 1420 may be placed on the skin away from the fistula, which may reduce the likelihood of infection. Skin button 1420 may be placed on the skin about 8 cm away from the fistula. Tether 1310 may be tunneled under the skin from the fistula to skin button 1420.

Skin button 1420 may include a base plate 1421. Base plate 1421 may have a bottom surface configured to be placed on the skin. Base plate 1421 may have a bottom surface with a soft material. Base plate 1421 may have an antimicrobial treatment. Base plate 1421 may be large enough to comfortably distribute forces from tether 1310.

Skin button 1420 may include a cleat 1422 through which tether 1310 may be passed. Cleat 1422 may include a channel 1423 with teeth configured to hold tether 1310.

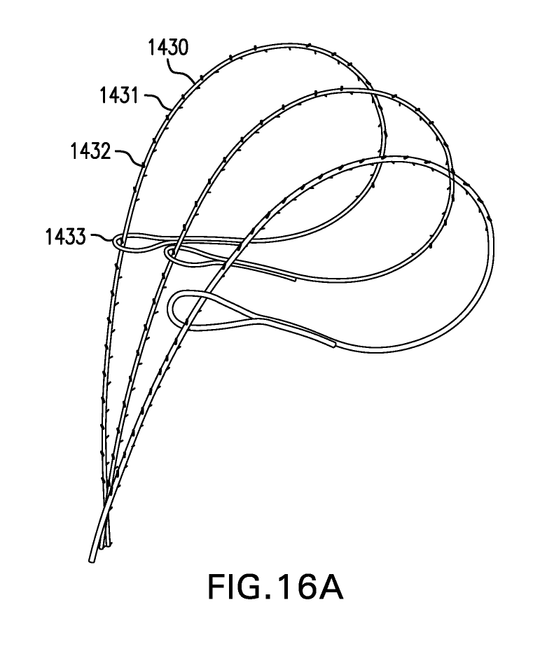

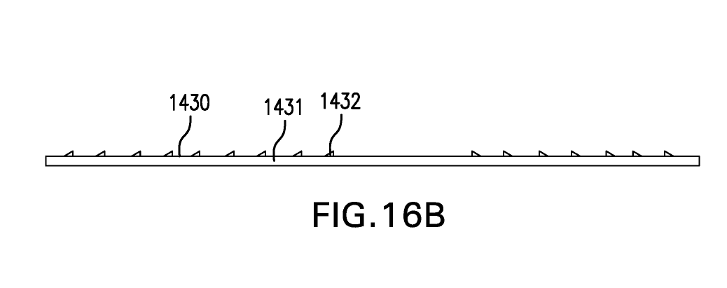

FIGS. 16A-16B show two embodiments of a barbed suture 1430. Barbed suture 1430 may include a suture 1431 having one or more barbs 1432. Barbed suture 1430 may have barbs 1432 along at least part of a length of suture 1431. Barbs 1432 may extend from suture 1431 at an angle. Barbs 1432 may allow suture 1431 to pulled easily through tissue in one direction, while providing high resistance to suture 1431 being pulled in an opposite direction.

Barbed suture 1430 may be bioabsorbable or non-absorbable. Barbed suture 1430 may have a suture size of one or more of 3-0, 2-0, 0, 1, and any other suitable size. Barbed suture 1430 may have a length of about 7 cm to about 20 cm. Barbed suture 1430 may include one or more of Covidien V-LOC™, Ethicon STRATAFIX™, and any other suitable barbed suture.

Barbed suture 1430 may include an antimicrobial coating. Antimicrobial coating may include triclosan and/or any other suitable antimicrobial agent.

FIG. 16A shows one embodiment of a barbed suture 1430. Barbed suture 1430 is unidirectional. Barbed suture 1430 may include a loop 1433.

FIG. 16B shows another embodiment of a barbed suture 1430. Barbed suture 1430 is bidirectional. Barbed suture 1430 may include a first portion 1430a and a second portion 1430b. Barbs 1432 from first portion 1430a and second portion 1430b may be angled toward each other. First portion 1430a and second portion 1430b may be inserted separately. First portion 1430a and second portion 1430b may have lengths that are the same or different.

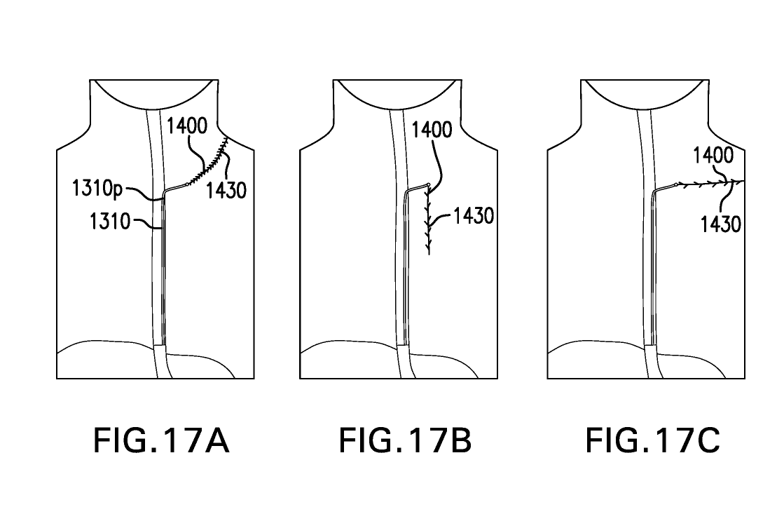

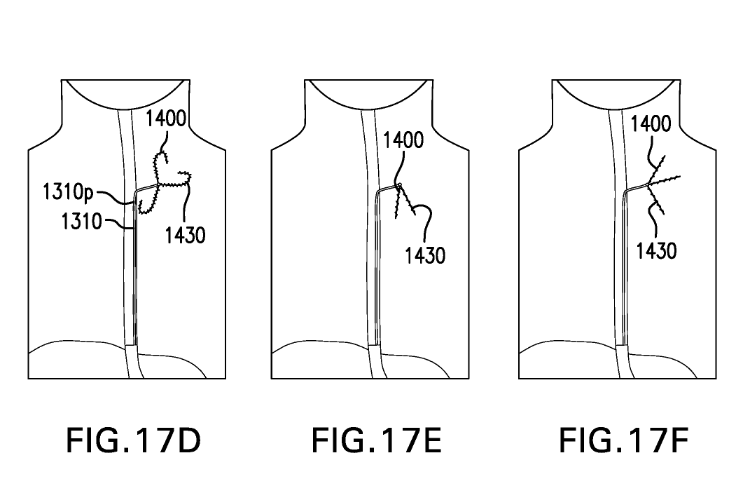

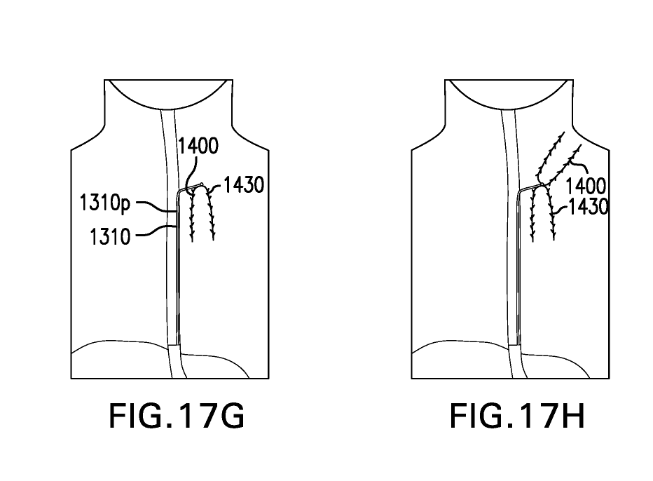

FIGS. 17A-17H show various embodiments of a tether anchor 1400 including at least one barbed suture 1430.

Barbed suture 1430 may be placed under the skin at or near where tether 1310 extends from the skin. Barbed suture 1430 may be inserted subcutaneously by tunneling under the skin with a needle. Barbed suture 1430 may be placed in one or more of a straight, U-shaped, J-shaped, and any other suitable pattern. Barbed suture 1430 may be coupled to proximal portion 1310p of tether 1310.

FIGS. 17A-17C show various embodiments of a tether anchor 1400 including a barbed suture 1430. Barbed suture 1430 may be unidirectional. Barbed suture 1430 may be placed proximally around the base of the neck, distally over the clavicle, or laterally along the clavicle.

FIGS. 17D-17F show various embodiments of a tether anchor 1400 including a plurality of barbed sutures 1430. Barbed sutures 1430 may be unidirectional.

FIGS. 17G-17H show two embodiments of a tether anchor 1400 including one or more barbed sutures 1430. Barbed sutures 1430 may be bidirectional.

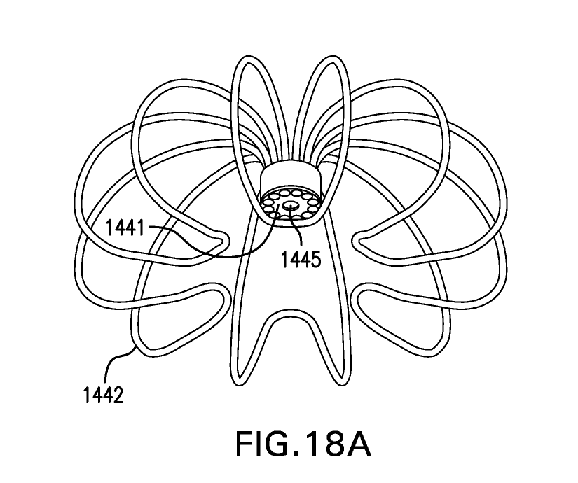

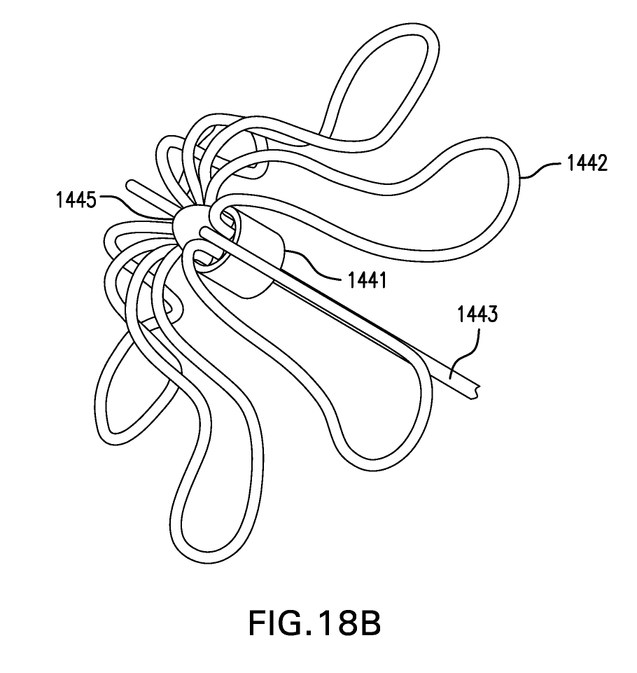

FIGS. 18A-18B show two embodiments of tether anchor 1400 including a fistula anchor 1440. Fistula anchor 1440 may be configured to be placed in a fistula between the esophagus E and the skin. Fistula anchor 1440 may have a collapsed configuration and an expanded configuration.

Fistula anchor 1440 may include a hub 1441. Hub 1441 may include a lumen 1445. Lumen 1445 may be configured to be coupled to tether 1310.

Fistula anchor 1440 may include one or more petals 1442. Petals 1442 may be coupled to hub 1441. Petals 1442 may be made of wire.

Fistula anchor 1440 may include a suture 1443. Suture 1443 may be coupled to hub 1441. Suture 1443 may be placed through lumen 1445 of hub 1441. Suture 1443 may allow fistula anchor 1443 to be coupled to tether 1310.

Fistula anchor 1440 may include one or more retention elements described in U.S. Patent Application Publication No. 2009/0012541, and distal retention elements of tissue anchors described in U.S. Pat. No. 9,675,489, which are incorporated by reference in their entireties.

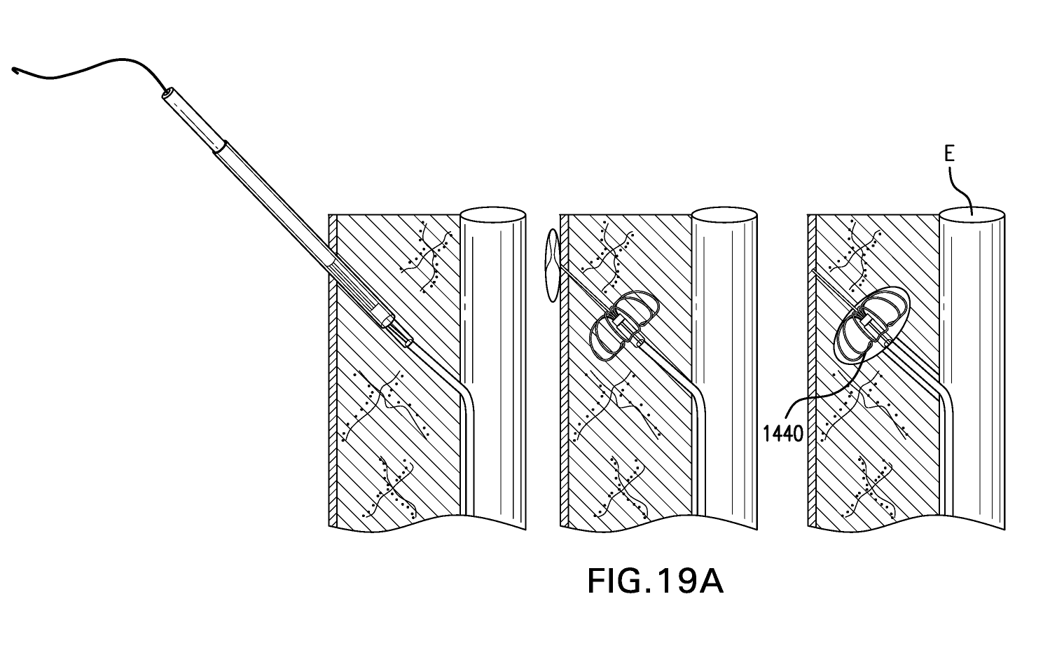

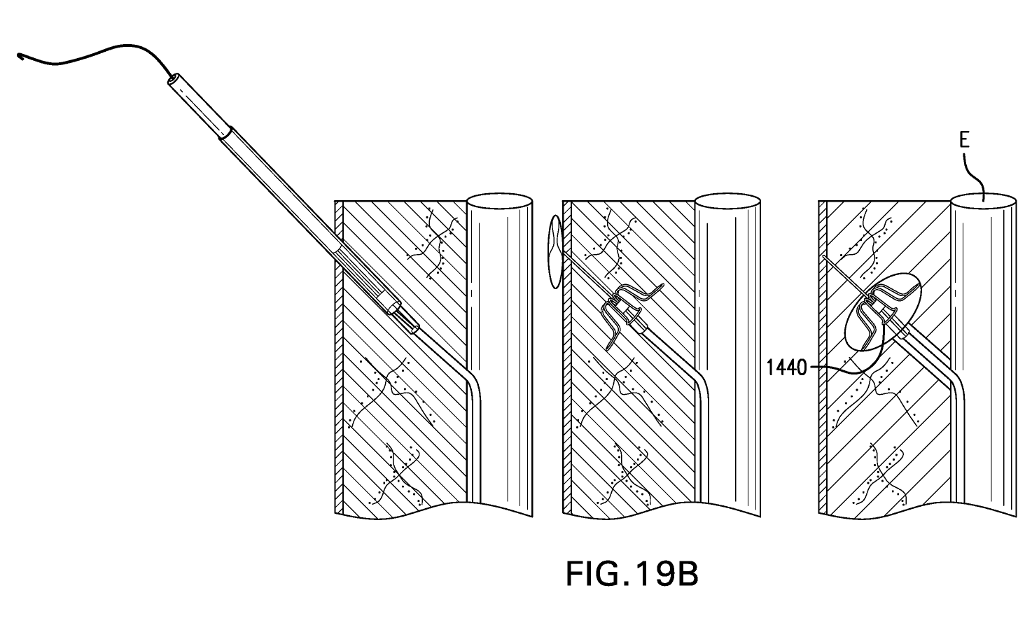

FIGS. 19A-19B show tether anchor 1400 including a fistula anchor 1440 placed in a fistula. Fistula anchor 1440 may be delivered with a catheter or a needle.

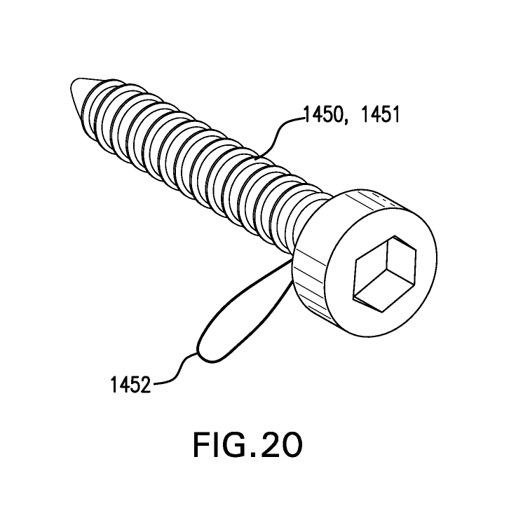

FIG. 20 shows one embodiment of a tether anchor 1400 including a bone attachment 1450. Bone attachment 1450 may be configured to be coupled to a bone. Bone attachment 1450 may be configured to be coupled to one or more of a clavicle, rib, mandible and any other suitable bone. Bone attachment 1450 may include a screw 1451. Bone attachment 1450 may include a tether coupling 1452. Tether coupling 1452 may allow tether 1310 to be coupled to bone attachment 1450. Tether coupling 1452 may include an eyelet and/or other suitable feature.

Proximal portion 1310p of tether 1310 or a suture coupled to proximal portion 1310p of tether may be inserted subcutaneously to bone attachment 1450.

FIG. 21 shows one embodiment of a tether anchor 1400 including a tooth attachment 1460. Tooth attachment 1460 may be configured to be coupled to a tooth. Tooth attachment 1460 may be configured to be coupled to a molar and/or any other tooth. Tooth attachment 1460 may include a band 1461. Tooth attachment 1460 may include a tether coupling 1462. Tether coupling 1462 may allow tether 1310 to be coupled to tooth attachment 1460. Tether coupling 1462 may include one or more of a post, stud, suture, and any other suitable device.

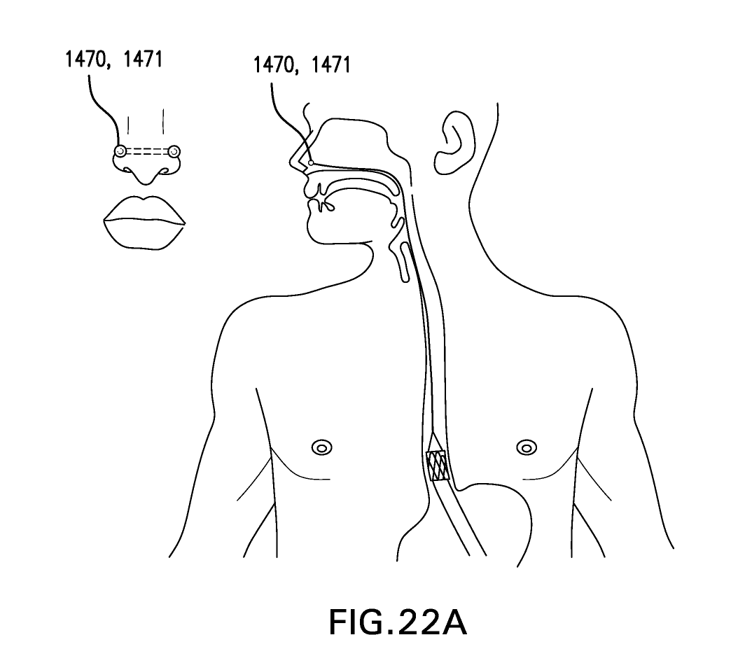

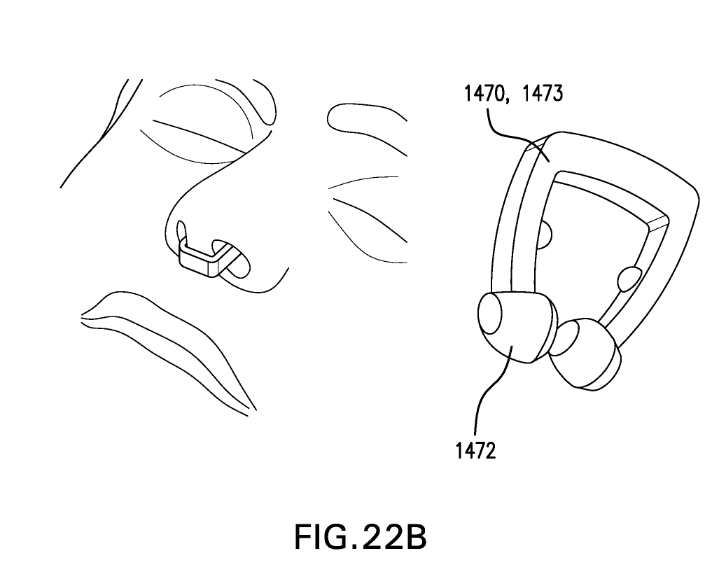

FIGS. 22A-22B show various embodiments of a tether anchor 1400 including a nasal septum attachment 1470. Nasal septum attachment 1470 may be configured to be coupled to the nasal septum. Nasal septum attachment 1470 may include a piercing 1471. Nasal septum attachment 1470 may include a septal clip 1473. Nasal septum attachment 1470 may include a tether coupling 1472. Tether coupling 1472 may allow tether 1310 to be coupled to nasal septum attachment 1470. Tether coupling 1472 may include one or more of a post, stud, suture, and any other suitable device.

FIG. 22A shows one embodiment of a tether anchor 1400 including a nasal septum attachment 1470. Nasal septum attachment 1470 may include a piercing 1471. Piercing 1471 may be placed through the nasal septum. Piercing 1471 may have ends that are hidden in the nostrils, protrude from the nostrils, or protrude from an exterior of the nose.

FIG. 22B shows another embodiment of a tether anchor 1400 including a nasal septum attachment 1470. Nasal septum attachment 1470 may include a septal clip 1473. Septal clip 1473 may be placed around the bottom of the nasal septum. Tether 1310 may be coupled to one side of septal clip 1473, or tether 1310 may be bifurcated and coupled to both sides of septal clip 1473.

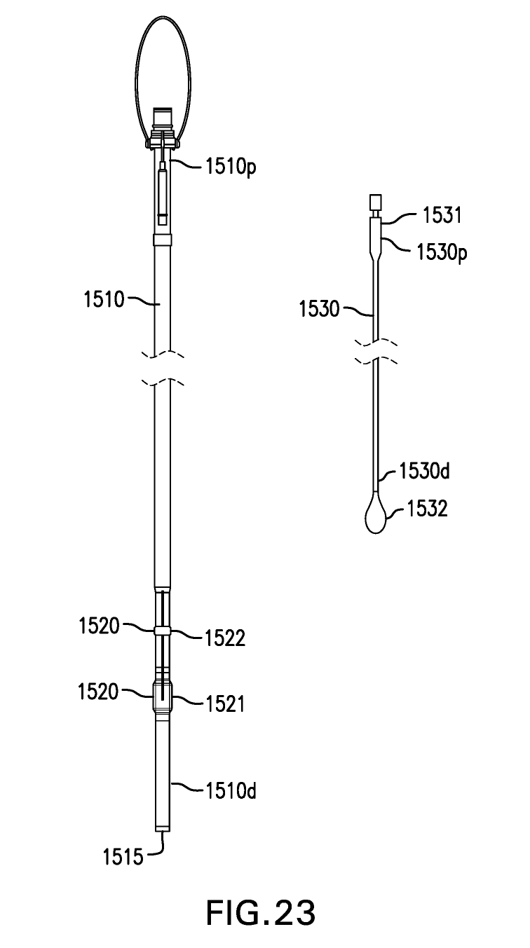

FIG. 23 shows one embodiment of a sleeve delivery device 1500. Sleeve delivery device 1500 may be configured to place at least a portion of sleeve 1120 in the intestine.

Sleeve delivery device 1500 may include a catheter 1510. Catheter 1510 may include a proximal portion 1510p and a distal portion 1510d. Catheter 1510 may include a lumen 1515. Lumen 1515 may be configured to accommodate an endoscope or other instrument. Catheter 1510 may have a width that is uniform or varying along its length. Catheter 1510 may have a width of about 10 mm to about 20 mm.

Sleeve delivery device 1500 may include one or more sealing elements 1520. Sealing elements 1520 may be formed on and/or coupled to an outside of catheter 1510. Sealing elements 1520 may be at or near distal portion 1510d of catheter 1510.

Sealing elements 1520 may be configured to create and/or help create a seal with cuff 1110 when cuff 1110 is placed over sealing elements 1520. Sealing elements 1520 may be configured to create a seal between an inside of cuff 1110 and an outside of catheter 1510.

Sealing elements 1520 may include a balloon 1521. Balloon 1521 may be coupled around distal portion 1510d of catheter 1510. Balloon 1521 may create a seal between cuff 1110 and catheter 1510 when cuff 1110 is placed over balloon 1521 and balloon 1521 is inflated.

Sealing elements 1520 may include at least one protrusion 1522. Protrusion 1522 may be proximal to balloon 1521. Protrusion 1522 may be circumferential. Protrusion 1522 may include one or more of a ring, bump, and any other suitable structure. Protrusion 1522 may prevent cuff 1110 from sliding off distal portion 1510d of catheter 1510 when drawstring 1113 of cuff 1110 is cinched proximal to protrusion 1522.

Sleeve delivery device 1500 may include a sleeve snare 1530. Sleeve snare 1530 may include a proximal portion 1530p and a distal portion 1530d. Proximal portion 1530p of sleeve snare 1530 may include a control handle 1531. Distal portion 1530d of sleeve snare 1530 may include a snare loop 1532.

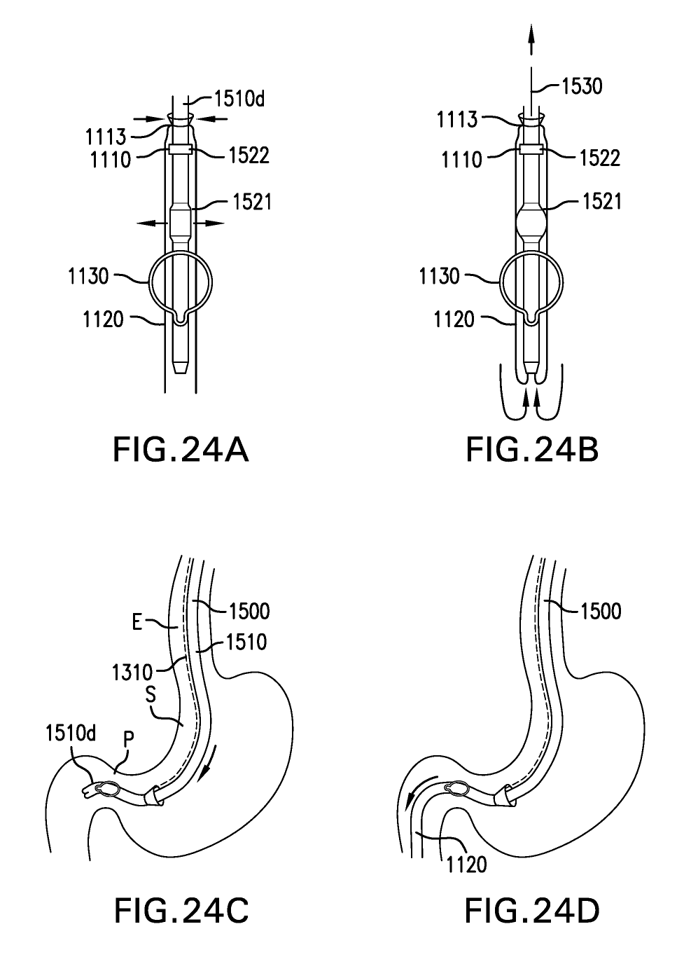

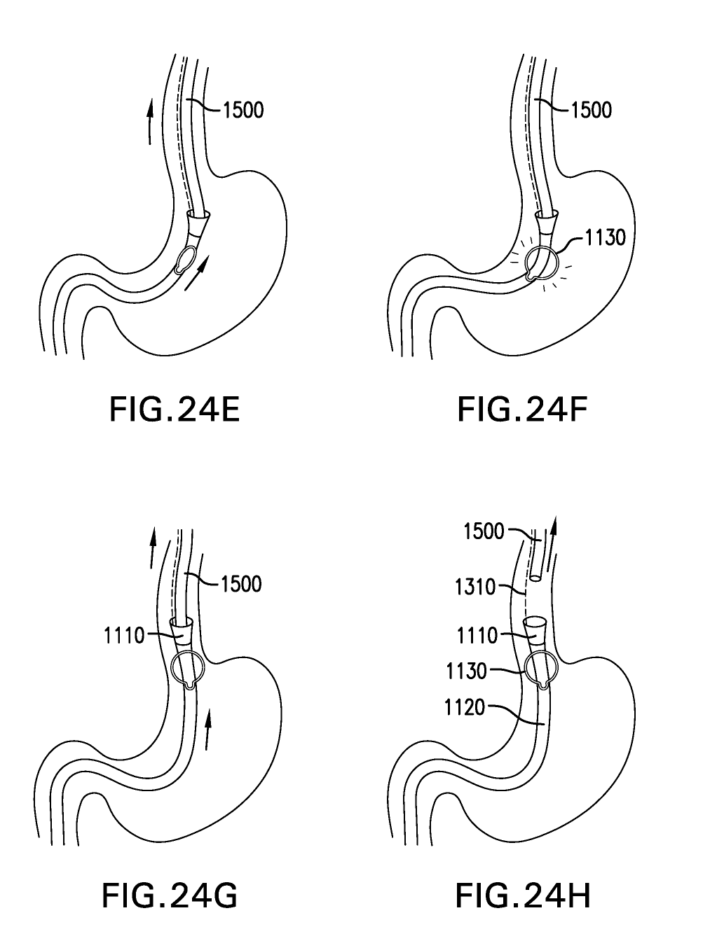

FIGS. 24A-24H show one embodiment of a method for delivering cuff-sleeve assembly 1100.

FIG. 24A shows loading cuff 1110 and sleeve 1120 onto catheter 1510. Cuff 1110, a portion of sleeve 1120, and gastric retainer 1130 are placed around distal portion 1510d of catheter 1510. Drawstring 1113 of cuff 1110 is cinched around catheter 1510 proximal to protrusion 1522. Balloon 1521 is inflated inside cuff 1110. Gastric retainer 1130 is constrained in a delivery configuration.

FIG. 24B shows inverting a remainder of sleeve 1120 into catheter 1510. Sleeve snare 1530 is passed through lumen 1515 of catheter 1510 and lumen 1105 of cuff-sleeve assembly 1100. Sleeve snare 1530 is coupled to and seals distal portion 1120d of sleeve 1120. Sleeve snare 1530 pulls distal portion 1120d of sleeve 1120 into lumen 1515 of catheter 1510 to invert sleeve 1120 into lumen 1515 of catheter 1510. An endoscope is passed through lumen 1515 of catheter 1510 inside inverted sleeve 1120.

FIG. 24C shows advancing catheter 1510 into the intestine. Catheter 1510 is advanced through the pylorus P to position distal portion 1510d of catheter 1510 in the proximal duodenum. Tether 1310 extends out of the mouth. Control lines for drawstring 1113 and gastric retainer 1130 extend out of the mouth.

FIG. 24D shows everting sleeve 1120 into the intestine. Endoscope is removed from lumen 1515 of catheter 1510. Control snare 1530 is coupled to and seals distal portion 1120d of sleeve 1120. Fluid is pumped through lumen 1515 of catheter 1510 to evert sleeve 1120 into the intestine. Control snare 1530 may be used to control the rate at which sleeve 1120 is everted. An endoscope and/or a pusher tool may be used through lumen 1515 of catheter 1510 to evert sleeve 1120 into the intestine. Control snare 1530 is uncoupled from distal portion 1120d of sleeve 1120.

FIG. 24E shows positioning catheter 1510 in the stomach S. Catheter 1510 is pulled back to position gastric retainer 1130 in the stomach S.

FIG. 24F shows deploying gastric retainer 1130. Gastric retainer 1130 is unconstrained and allowed to open in the stomach S.

FIG. 24G shows positioning cuff 1110 in the esophagus E. Catheter 1510 is pulled back to position cuff 1110 in the esophagus E.

FIG. 24H shows withdrawing catheter 1510. Balloon 1521 is deflated. Drawstring 1113 of cuff 1110 is uncinched. Catheter 1510 is withdrawn from the esophagus E. Proximal portion 1310p of tether 1310 may be secured with tooth attachment 1460 and/or nasal septum attachment 1470, or tether 1310 may be placed through a fistula between the esophagus and the skin, as described below.

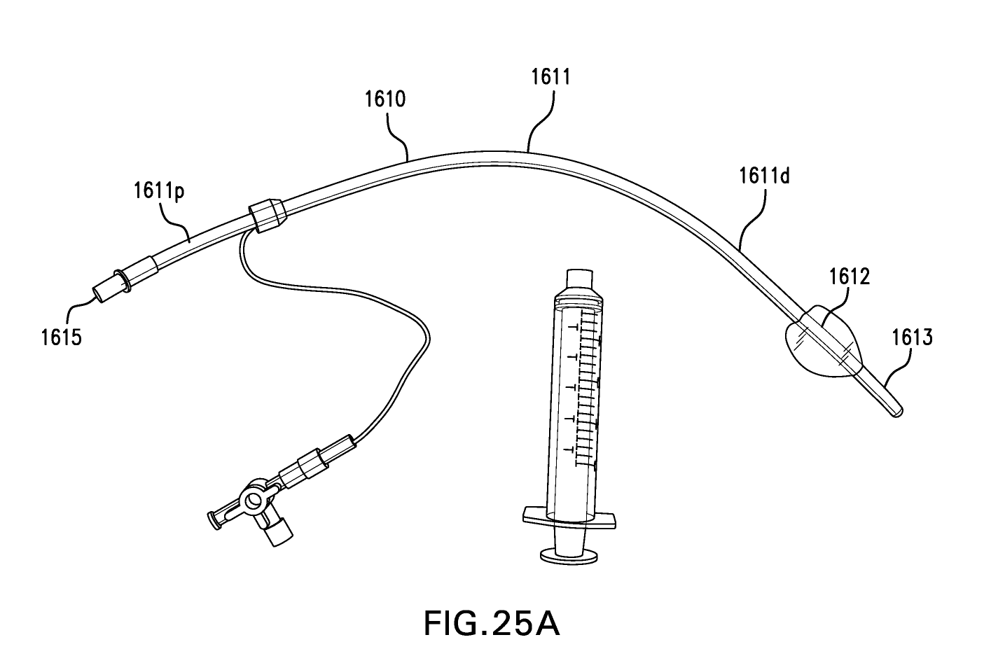

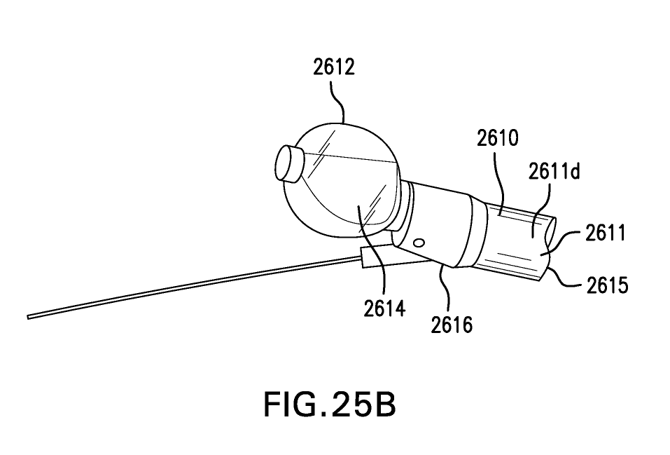

FIG. 25A shows one embodiment of a balloon catheter 1610. Balloon catheter 1610 may include a catheter 1611. Catheter 1611 may include a proximal portion 1611p and a distal portion 1611d. Catheter 1611 may be echoic. Catheter 1611 may include a lumen 1615. Lumen 1615 may be configured to receive an endoscope. Catheter 1611 may include one or more depth markings 1619. Catheter 1611 may have a length of about 25 cm to about 60 cm.

Balloon catheter 1610 may include a balloon 1612. Balloon 1612 may be coupled at or near distal portion 1610d of catheter 1610. Balloon 1612 may be anechoic. Balloon 1612 may be inflated with a fluid, such as saline or water. Balloon 1612 may have a width of about 25 mm when inflated. Balloon 1612 may have a length of about 25 mm to about 50 mm when inflated. Balloon 1612 may be made of a material that does not burst when pierced by a needle.

Balloon catheter 1610 may include a light 1613. Light 1613 may be coupled to catheter 1610. Light 1613 may be configured to illuminate balloon 1612. Light 1613 may be directional, with the direction of light 1613 corresponding to circumferential markings on proximal portion 1611p of catheter 1611. Light 1613 may allow a position of balloon 1612 to be seen through the neck of a patient. Light 1613 may include optical fibers and/or an LED.

FIG. 25B shows another embodiment of a balloon catheter 2610.

Balloon catheter 2610 may include a catheter 2611. Catheter 2611 may include a proximal portion 2611p and a distal portion 2611d. Catheter 2611 may be echoic. Catheter 2611 may include a lumen 2615. Lumen 2615 may be configured to receive an endoscope.

Balloon catheter 2610 may include a balloon 2612. Balloon 2612 may be coupled at or near distal portion 2610d of catheter 2610. Balloon 2612 may be anechoic. Balloon 2612 may be inflated with a fluid, such as saline or water. Balloon 2612 may have a width of about 25 mm when inflated. Balloon 2612 may have a length of about 25 mm to about 50 mm when inflated. Balloon 2612 may be made of a material that does not burst when pierced by a needle.

Balloon catheter 2610 may include a camera 2614. Camera 2614 may be coupled to catheter 2610. Camera 2614 may include fiber optics and/or a miniaturized camera.

Balloon catheter 2610 may include an ultrasound array 2616. Ultrasound array 2616 may be coupled to catheter 2610. Ultrasound array 2616 may include a linear ultrasound array.

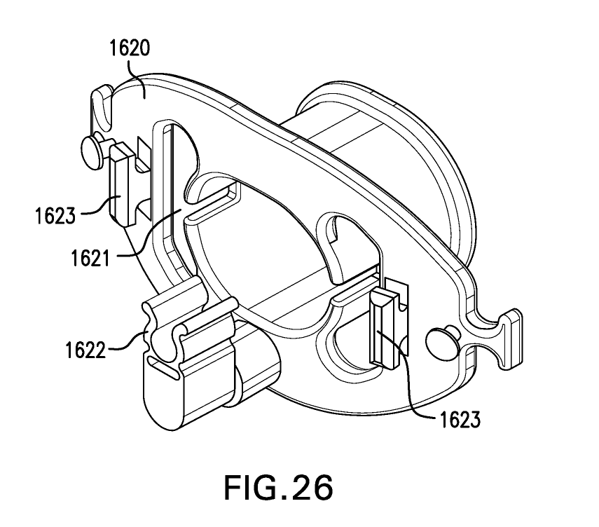

FIG. 26 shows one embodiment of a bite block 1620. Bite block 1620 may separate the jaws of a patient so that devices and instruments may be inserted through the mouth. Bite block 1620 may protect the teeth of a patient.

Bite block 1620 may include one or more openings 1621 through which devices and instruments may be passed. Bite block 1620 may include a clip 1622 configured to be coupled to balloon catheter 1610. Bite block 1620 may include one or more cleats 1623 configured to be coupled to tether 1310 and/or a control line for drawstring 1113 of cuff 1110. Bite block 1620 may be used with a strap.

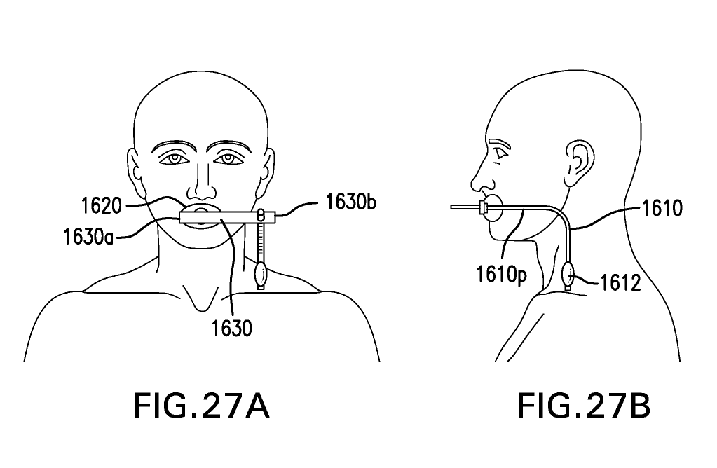

FIGS. 27A-27B show one embodiment of a balloon depth arm 1630. Balloon depth arm 1630 may have a first end 1630a and a second end 1630b. First end 1630a of balloon depth arm 1630 may be configured to be coupled to bite block 1620. Second end 1630b of balloon depth arm 1630 may be coupled to proximal portion 1610p of balloon catheter 1610. Balloon depth arm 1630 may extend to a side of bite block 1620. Balloon depth arm 1630 may allow a distance from the mouth to where balloon 1612 is to be advanced in the esophagus E to be estimated.

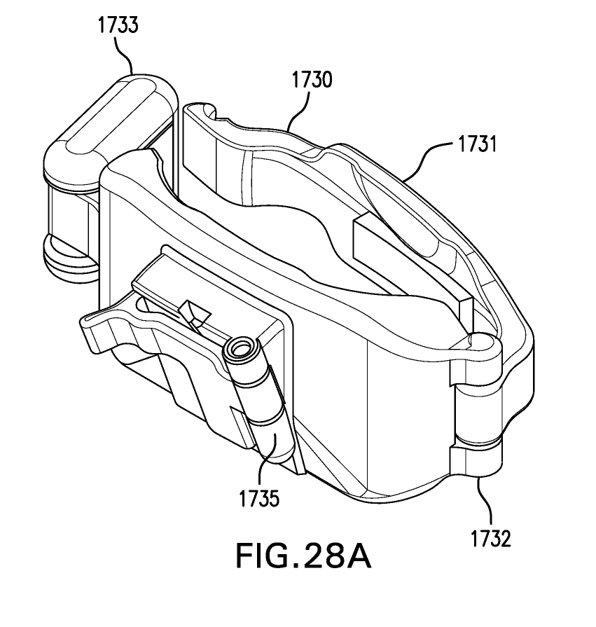

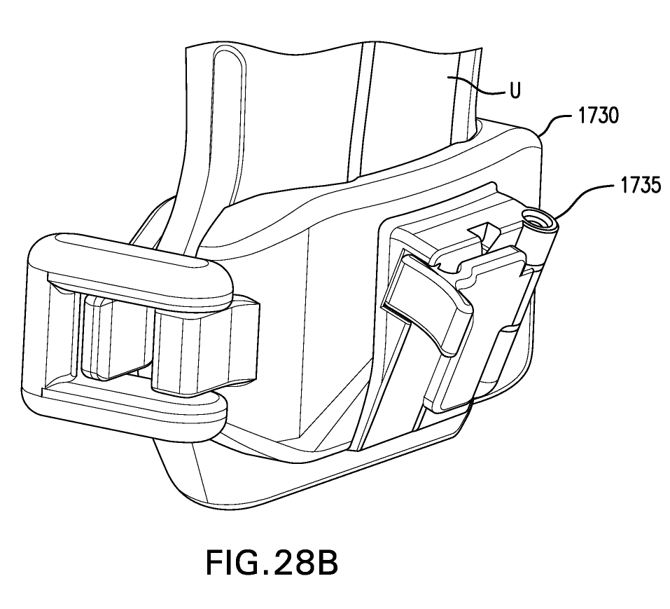

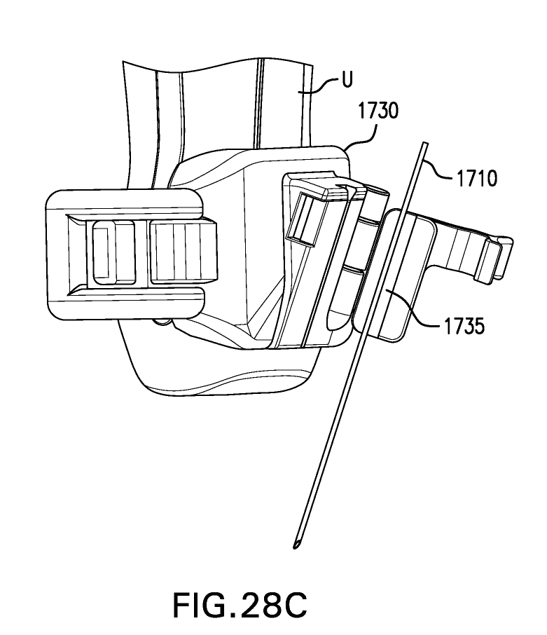

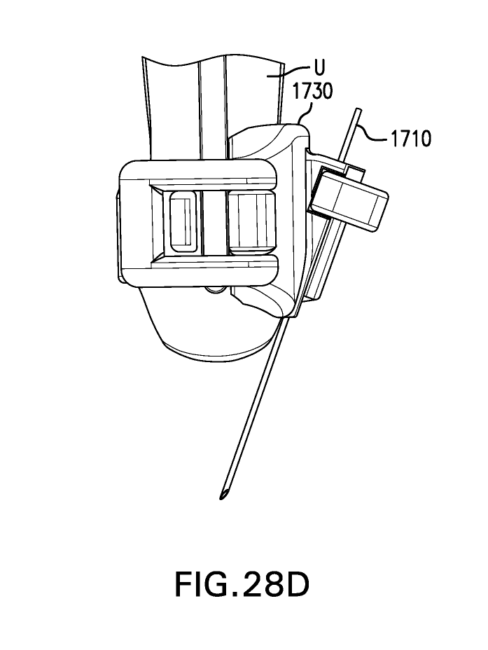

FIGS. 28A-28D show one embodiment of a needle guide 1730. Needle guide 1730 may include a probe coupling 1731. Probe coupling 1731 may include a hinge 1732 and a latch 1733. Probe coupling 1731 may be configured to be coupled to an ultrasound probe.

Needle guide 1730 may include a guide channel 1735. Guide channel 1735 may be configured to receive a needle 1710. Guide channel 1735 may be configured to direct needle 1710 in an out-of-plane orientation. Guide channel 1735 may be configured to direct needle 1710 under one of the longer sides of an ultrasound probe, as opposed to one of the narrower ends. Guide channel 1735 may be configured to direct needle 1710 at an angle of about 70 degrees to about 80 degrees to a plane tangent to the center of the skin-contacting face of an ultrasound probe. Guide channel 1735 may be configured to direct needle 1710 through a point about 3 cm to about 4 cm beneath the center skin-contacting face of the ultrasound probe. Guide channel 1735 may be configured to limit a depth needle 1710 may be advanced.

FIG. 28A shows needle guide 1730. FIG. 28B shows needle guide 1730 coupled to an ultrasound probe U. FIG. 28C shows needle guide 1730 with guide channel 1735 open to receive needle 1710. FIG. 28D shows needle guide 1730 with needle 1730 in guide channel 1735.

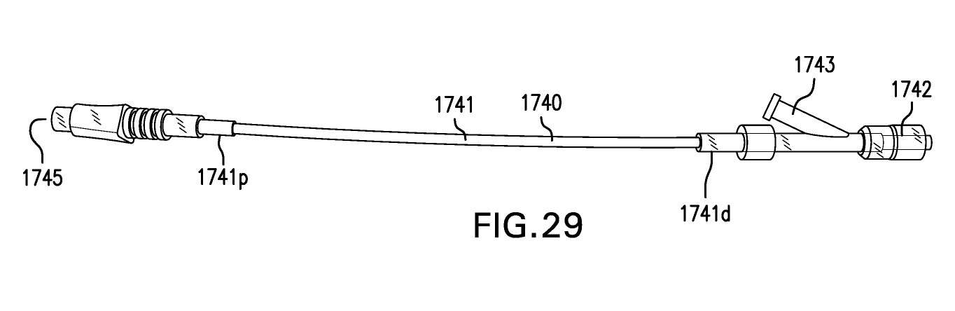

FIG. 29 shows one embodiment of a guidewire holder 1740. Guidewire holder 1740 may facilitate advancing of guidewire 1720 through needle 1710.

Guidewire holder 1740 may include a body 1741 having a proximal portion 1741p and a distal portion 1741d. Body 1741 may include a lumen 1745. Lumen 1745 may be configured to receive guidewire 1720. Lumen 1745 may receive guidewire 1720 through proximal portion 1741p of body 1741. Lumen 1745 may be configured to prevent guidewire 1720 from falling out of lumen 1745, yet allow guidewire 1720 to be advanced freely through lumen 1745. Body 1741 may be made of a translucent material. Body 1741 may have a length of about 20 cm.

Guidewire holder 1740 may include a needle connector 1742 coupled to distal portion 1741d of body 1741. Needle connector 1742 may be configured to be coupled to needle 1710. Needle connector 1742 may include a luer slip or luer lock.

Guidewire holder 1740 may include a side port 1743. Side port 1743 may be formed at or near distal portion 1741d of body 1741. Side port 1743 may be formed proximal to needle connector 1742. Side port 1743 may allow fluid through needle 1710 to escape. Side port 1743 may be coupled to a syringe to apply a vacuum to needle 1710.

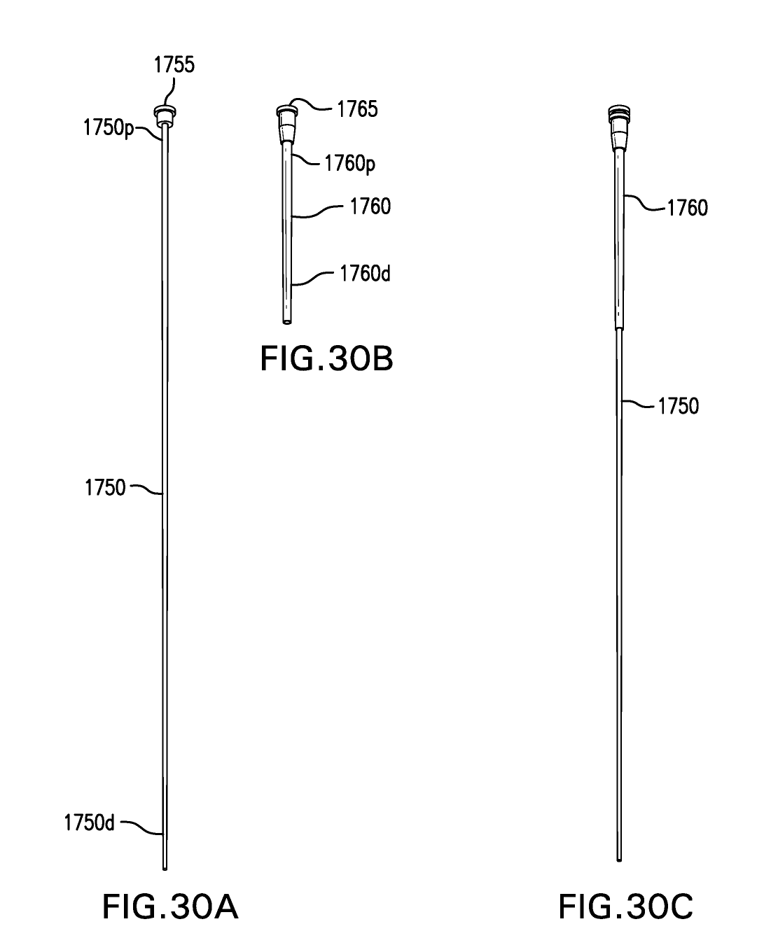

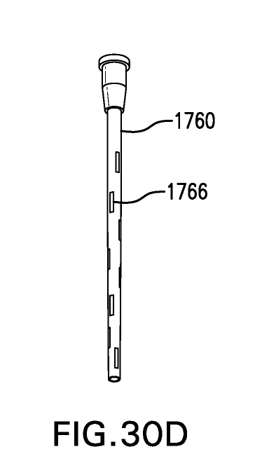

FIG. 30A shows one embodiment of a dilator 1750. FIG. 30B shows one embodiment of a sheath 1760. FIG. 30C shows dilator 1750 placed in lumen 1765 of sheath 1760.

Dilator 1750 may include a proximal portion 1750p and a distal portion 1750d. Dilator 1750 may include a lumen 1755. Dilator 1750 may have a thickness which is about the same as a thickness of tether 1310.

Sheath 1760 may include a proximal portion 1760p and a distal portion 1760d. Sheath 1760 may include a lumen 1765 which is sufficiently large to allow dilator 1750 and tether 1310 to pass through. Sheath 1760 may include a side port which allows lumen 1765 to be flushed.

FIG. 30D shows another embodiment of a sheath 1760. Sheath 1760 may include one or more openings 1766 which allow tissue surrounding sheath 1760 to be flushed.

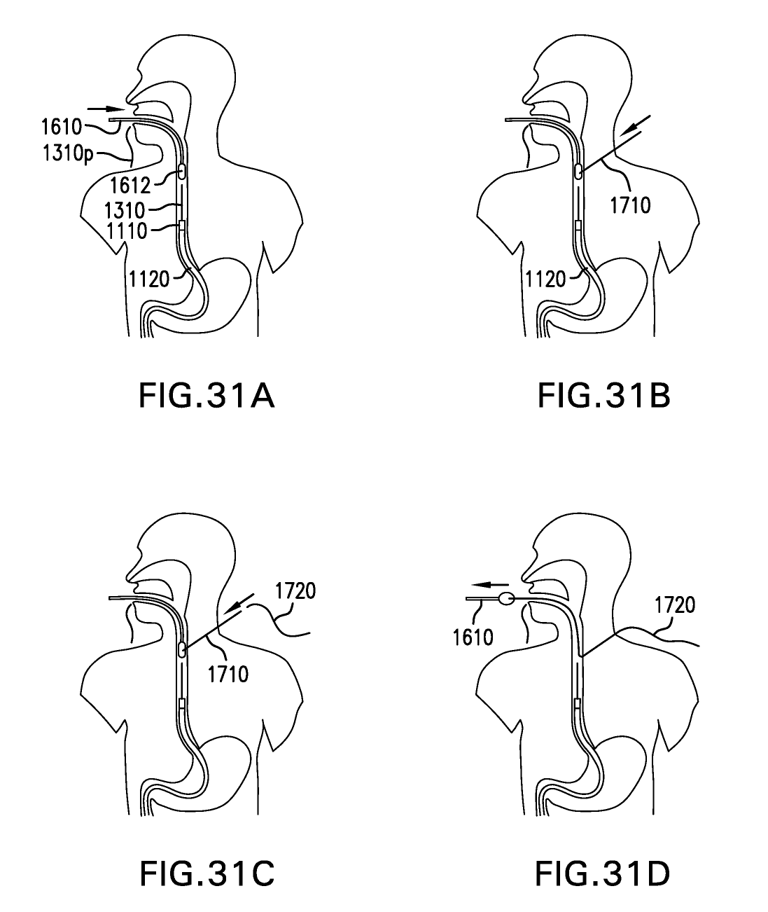

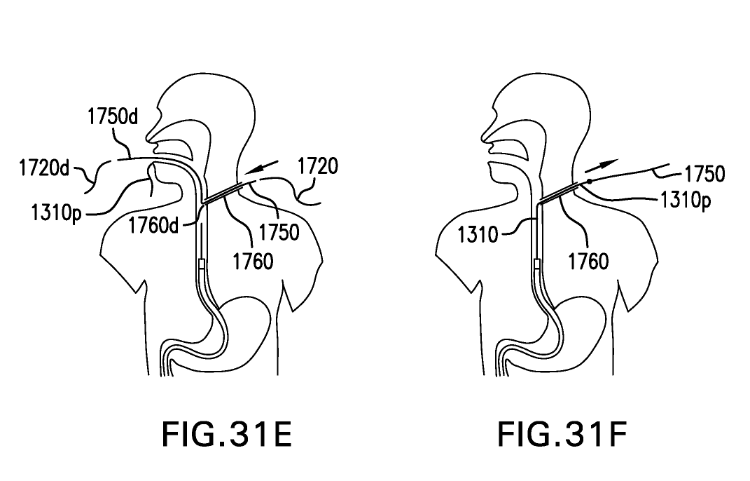

FIGS. 31A-31F show one embodiment of a method for placing tether 1310.

FIG. 31A shows advancing a balloon catheter 1610 into the esophagus E. Cuff 1110 and sleeve 1120 have been placed, and tether 1310 extends out of the mouth. Balloon catheter 1610 is advanced through the mouth into the esophagus E. Balloon 1612 may be centered just proximal of the upper margin of the left sternoclavicular joint. Balloon 1612 is inflated with water. Balloon 1612 may move the esophagus E away from adjacent anatomy to create an access path from the skin to the esophagus E. Bite block 1620 may be used to hold tether 1310 and balloon catheter 1610.

FIG. 31B shows advancing a needle 1710 into balloon 1612. An ultrasound probe U may be placed transversely on the neck and centered on balloon 1612. The ultrasound probe U may be pressed toward balloon 1612 to reduce a distance between the skin and the esophagus E. Needle 1710 is advanced through the skin, through the esophageal wall, and into balloon 1612 to create a fistula between the esophagus E and the skin. Needle 1710 may be advanced using a needle guide 1730 coupled to the ultrasound probe U. Needle 1710 may enter the skin at the base of the left side of the neck, just above the left sternoclavicular joint. Water may flow out of needle 1710 when needle 1710 pierces balloon 1612. Color Doppler mode may be used to avoid adjacent vascular anatomy such as the common carotid artery and internal jugular vein. Needle 1710 may have a length of about 7 cm. Needle 1710 may have a thickness of about 20 gauge to about 22 gauge.

FIG. 31C shows advancing a guidewire 1720 into balloon 1612. Guidewire 1720 is advanced through needle 1710. Guidewire holder 1740 may be used to thread guidewire 1720 through needle 1710. Tip 1729 of guidewire 1720 is advanced into balloon 1612. Tip 1729 of guidewire 1720 may be configured to resist removal from balloon 1612. Tip 1729 may be floppy and/or include a pigtail. Tip 1729 may be curled within balloon 1612. Guidewire 1720 may have a length of about 50 cm to about 130 cm. Tip 1729 may have a length of about 4 cm to about 10 cm. Needle 1710 is withdrawn and guidewire 1720 is left in place.

FIG. 31D shows withdrawing balloon catheter 1610 from the esophagus E. Balloon 1612 is deflated, and balloon catheter 1610 is withdrawn from the esophagus E. Balloon 1612 holds tip 1729 of guidewire 1720 and brings distal portion 1720d of guidewire 1720 out of the mouth.

FIG. 31E shows advancing a dilator 1750 over guidewire 1720. Dilator 1750 is advanced over guidewire 1720 through the fistula. Dilator 1750 is advanced to extend distal portion 1750d of dilator 1750 out of the mouth. Dilator 1750 may have a length of about 35 cm to about 45 cm.

Optionally, a sheath 1760 may be advanced with dilator 1750 through the fistula. Sheath 1760 may be advanced to extend distal portion 1760d of sheath 1760 into the esophagus E. Sheath 1760 may have a length of about 5 cm to about 15 cm.

Guidewire 1720 is withdrawn and dilator 1750 is left in place. If sheath 1760 was used, sheath 1760 is also left in place.

FIG. 31F shows pulling tether 1310 through the fistula. Distal portion 1750d of dilator 1750 is coupled to proximal portion 1310p of tether 1310. Distal portion 1750d of dilator 1750 may be coupled to proximal portion 1310p of tether 1310 with one or more of a knot, threaded coupling, press-fit mechanical coupling, and any other suitable device or method. Dilator 1750 is withdrawn through the fistula to pull proximal portion 1310p of tether 1310 through the fistula and out of the skin. If sheath 1760 was used, dilator 1750 is withdrawn through sheath 1760 to pull proximal portion 1310p of tether 1310 through sheath 1760 and out of the skin, and sheath 1760 is withdrawn. Proximal portion 1310p of tether 1310 is secured to an anchor site.

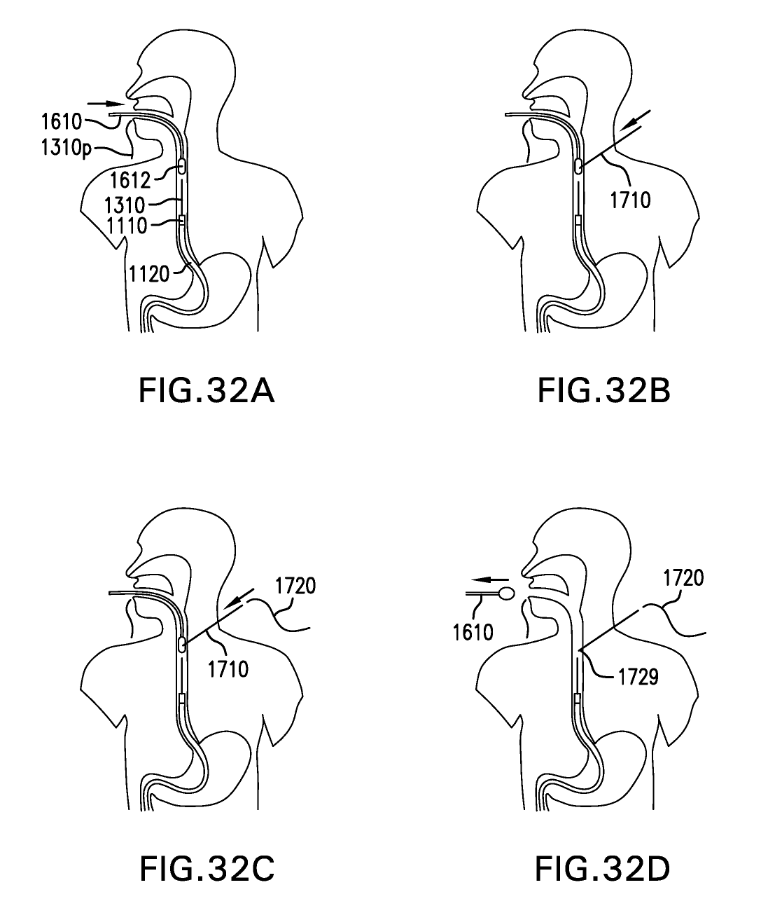

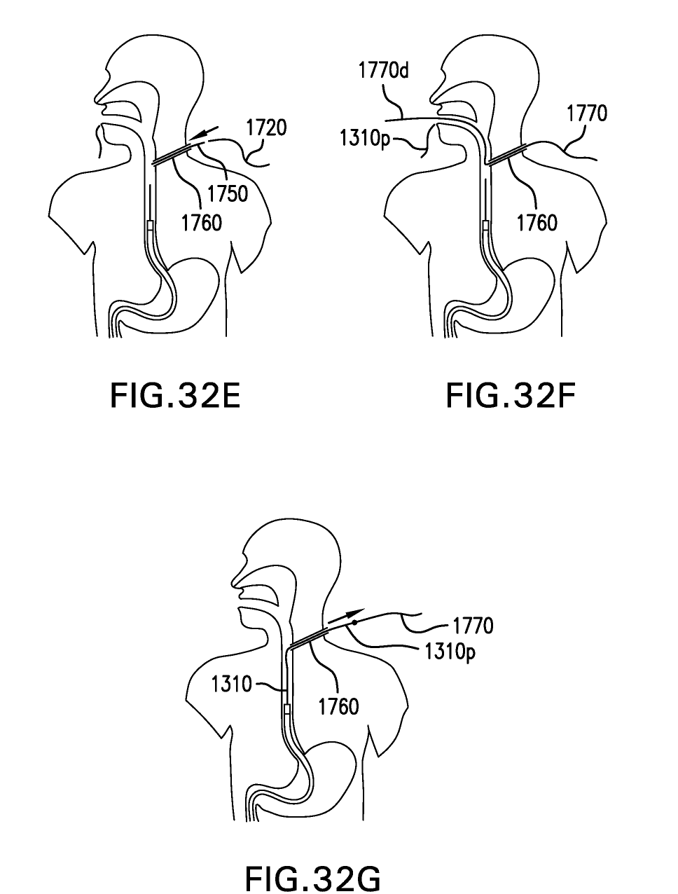

FIGS. 32A-32G show another embodiment of a method for placing tether 1310.

FIG. 32A shows advancing a balloon catheter 1610 into the esophagus E. Cuff 1110 and sleeve 1120 have been placed, and tether 1310 extends out of the mouth. Balloon catheter 1610 is advanced through the mouth into the esophagus E. Balloon 1612 may be centered just proximal of the upper margin of the left sternoclavicular joint. Balloon 1612 is inflated with water. Balloon 1612 may move the esophagus E away from adjacent anatomy to create an access path from the skin to the esophagus E. Bite block 1620 may be used to hold tether 1310 and balloon catheter 1610.

FIG. 32B shows advancing a needle 1710 into balloon 1612. An ultrasound probe U may be placed transversely on the neck and centered on balloon 1612. The ultrasound probe U may be pressed toward balloon 1612 to reduce a distance between the skin and the esophagus E. Needle 1710 is advanced through the skin, through the esophageal wall, and into balloon 1612 to create a fistula between the esophagus E and the skin. Needle 1710 may be advanced using a needle guide 1730 coupled to the ultrasound probe U. Needle 1710 may enter the skin at the base of the left side of the neck, just above the left sternoclavicular joint. Water may flow out of needle 1710 when needle 1710 pierces balloon 1612. Color Doppler mode may be used to avoid adjacent vascular anatomy such as the common carotid artery and internal jugular vein. Needle 1710 may have a length of about 7 cm. Needle 1710 may have a thickness of about 20 gauge to about 22 gauge.

FIG. 32C shows advancing a guidewire 1720 into balloon 1612. Guidewire 1720 is advanced through needle 1710. Guidewire holder 1740 may be used to thread guidewire 1720 through needle 1710. Tip 1729 of guidewire 1720 is advanced into balloon 1612. Guidewire 1720 may have a length of about 35 cm to about 45 cm. Needle 1710 is withdrawn and guidewire 1720 is left in place.

FIG. 32D shows withdrawing balloon catheter 1610 from the esophagus E. Balloon 1612 is deflated, and balloon catheter 1610 is withdrawn from the esophagus E.

FIG. 32E shows advancing a dilator 1750 and a sheath 1760 over guidewire 1720. Dilator 1750 and sheath 1760 are advanced over guidewire 1720 through the fistula. Dilator 1750 and sheath 1760 are advanced to extend distal portion 1750d of dilator 1750 and distal portion 1760d of sheath 1760 into the esophagus E. Dilator 1750 and sheath 1760 may each have a length of about 5 cm to about 15 cm.

Guidewire 1720 and dilator 1750 are withdrawn, and sheath 1760 is left in place.

FIG. 32F shows advancing suture 1770 through sheath 1760. Suture 1770 is advanced through sheath 1760 until suture 1770 extends into the esophagus E. An endoscopic grasper may then be used to bring distal portion 1770d of suture 1770 out of the mouth. Suture 1770 may have a length of about 50 cm to about 130 cm.

Alternatively, a guidewire 1720 with a suture 1770 coupled to a trailing end of guidewire 1720 may be advanced through sheath 1760 and up the esophagus E until suture 1770 extends out of the mouth. Guidewire 1720 may then be uncoupled from suture 1770 to leave suture 1770 in place.

FIG. 32G shows pulling tether 1310 through sheath 1760. Distal portion 1770d of suture 1770 is coupled to proximal portion 1310p of tether 1310. Distal portion 1770d of suture 1770 may be coupled to proximal portion 1310p of tether 1310 with one or more of a knot, threaded coupling, press-fit mechanical coupling, and any other suitable device or method. Suture 1770 is withdrawn through sheath 1760 to pull proximal portion 1310p of tether 1310 through sheath 1760 and out of the skin. Sheath 1760 is withdrawn. Proximal portion 1310p of tether 1310 is secured to an anchor site.

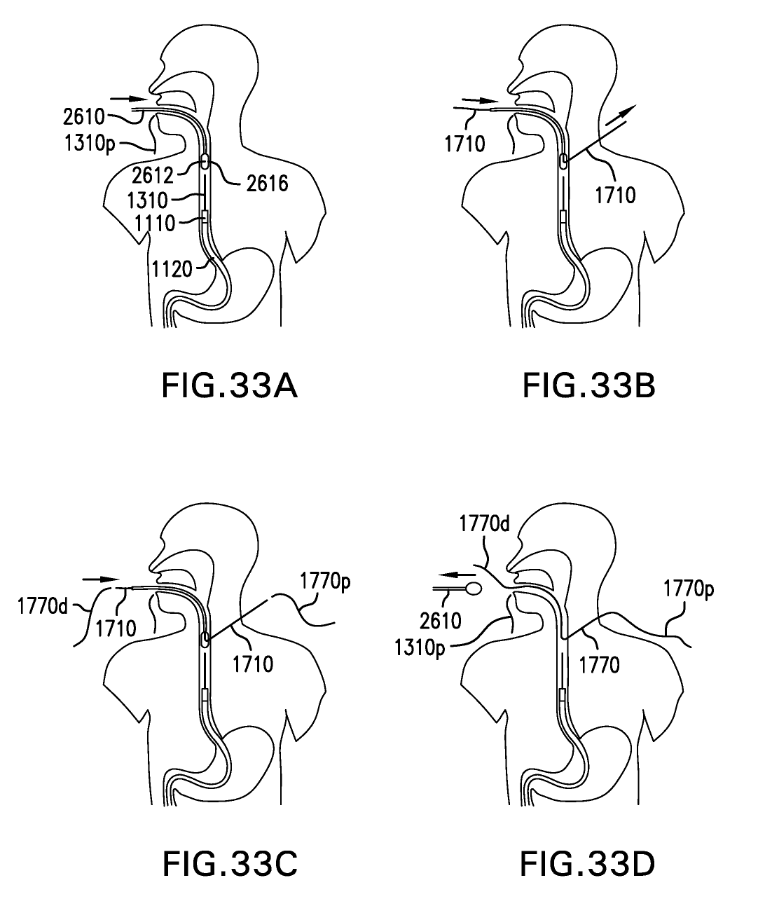

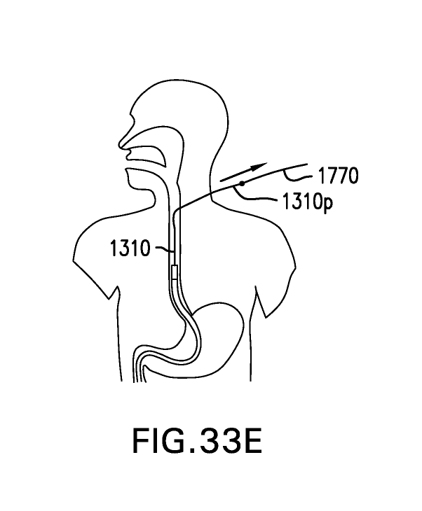

FIGS. 33A-33E show another embodiment of a method for placing tether 1310.

FIG. 33A shows advancing a balloon catheter 2610 into the esophagus E. Cuff 1110 and sleeve 1120 have been placed, and tether 1310 extends out of the mouth. Balloon catheter 2610 is advanced through the mouth into the esophagus E. Balloon 2612 may be centered just proximal of the upper margin of the left sternoclavicular joint. Balloon 2612 is inflated with a fluid, such as saline or water. Balloon 2612 may move the esophagus E away from adjacent anatomy to create an access path from the skin to the esophagus E. Balloon 2612 may increase harmonic coupling of ultrasound array 2616 and the surrounding tissue. Balloon 2612 may stretch surrounding tissue so that the tissue layers and any anomalies within them are more clearly visible. Bite block 1620 may be used to hold tether 1310 and balloon catheter 2610.

FIG. 33B shows advancing a needle 1710 out of the skin. Needle 1710 is advanced through working lumen 2615 of balloon catheter 2610. Needle 1710 is advanced through the esophageal wall and out through the skin to create a fistula between the esophagus E and the skin. Needle 1710 may exit the skin at the base of the left side of the neck, just above the left sternoclavicular joint. Color Doppler mode may be used to avoid adjacent vascular anatomy such as the common carotid artery and internal jugular vein. Needle 1710 may have a length of about 60 cm to about 120 cm. Needle 1710 may have a thickness of about 20 gauge to about 22 gauge.

FIG. 33C shows passing a suture 1770 through needle 1710. Suture 1770 is passed through needle 1710 until proximal portion 1770d of suture 1770 extends from the skin. A guidewire 1720 with a suture 1770 coupled to a trailing end of guidewire 1720 may be advanced through needle 1710 until suture 1770 extends from the skin. Guidewire 1720 may then be uncoupled from suture 1770. Suture 1770 may have a length of about 50 cm to about 130 cm. Needle 1710 is withdrawn and suture 1770 is left in place.

FIG. 33D shows withdrawing catheter 2610 from the esophagus E. Balloon 2612 is deflated, and balloon catheter 2610 is withdrawn from the esophagus E.

FIG. 33E shows pulling tether 1310 through the fistula. Distal portion 1770d of suture 1770 is coupled to proximal portion 1310p of tether 1310. Distal portion 1770d of suture 1770 may be coupled to proximal portion 1310p of tether 1310 with one or more of a knot, threaded coupling, press-fit mechanical coupling, and any other suitable device or method. Distal portion 1770d of suture 1770 may be coupled to proximal portion 1310p of tether 1310 with a device or method having a small thickness to reduce trauma to the fistula. Suture 1770 is withdrawn through the fistula to pull proximal portion 1310p of tether 1310 through the fistula and out of the skin. Proximal portion 1310p of tether 1310 is secured to an anchor site.

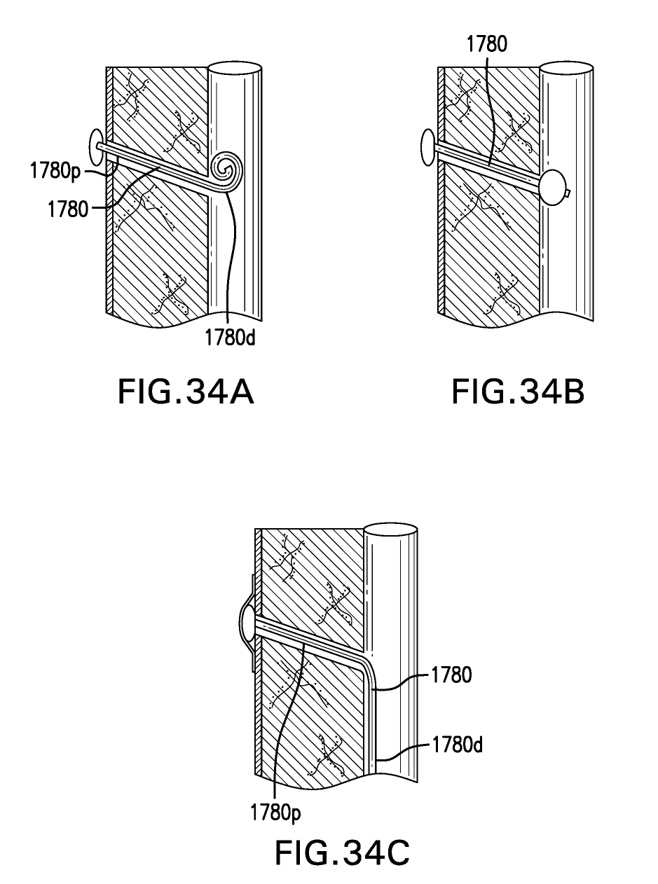

FIGS. 34A-34C show various embodiments of a temporary tube 1780. Temporary tube 1780 may have a proximal portion 1780p and a distal portion 1780d. Proximal portion 1780p of temporary tube 1780 may be configured to be coupled to an anchor site. Distal portion 1780d of temporary tube 1780 may be configured to resist removal from the esophagus. Distal portion 1780d of temporary tube 1780 may include a pigtail.

Temporary tube 1780 may be placed in the fistula without first delivering cuff 1110 and sleeve 1120. Distal portion 1780d of temporary tube 1780 may be placed in the esophagus E. After a period of time has passed to allow fistula to heal, cuff 1110 and sleeve 1120 may be delivered, temporary tube 1780 removed, and tether 1310 placed through the fistula.

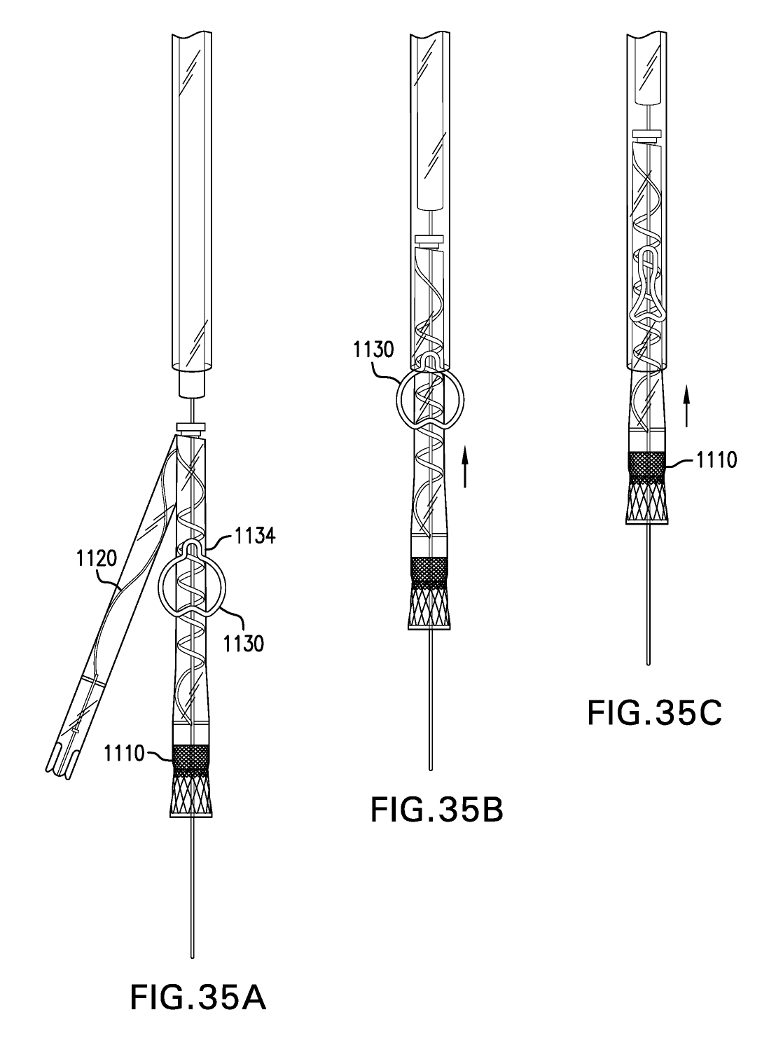

FIGS. 35A-35C show one embodiment of a method for removing cuff-sleeve assembly 1100.

FIG. 35A shows pushing cuff 1110 into the stomach S. Tether 1310 is cut and cuff 1110 is pushed into the stomach S. Tether 1310 may be cut at the skin or in the esophagus E.

FIG. 35B shows pulling gastric retainer 1130 into an overtube or the esophagus E. Gastric retainer 1130 may be pulled by tab 1134 using an endoscopic grasper. Gastric retainer 1130 may be turned upside down. Gastric retainer 1130 collapses into the overtube or the esophagus E.

FIG. 35C shows pulling cuff 1110 and sleeve 1120 into the overtube or esophagus E. Tether 1310 may be pulled out of the fistula.

Tether assembly 1300 and tether anchor 1400 may be used with other intraluminal devices placed in the gastrointestinal tract. Tether assembly 1300 and tether anchor 1400 may be used with a flow restriction device, an esophageal stent, and other devices.

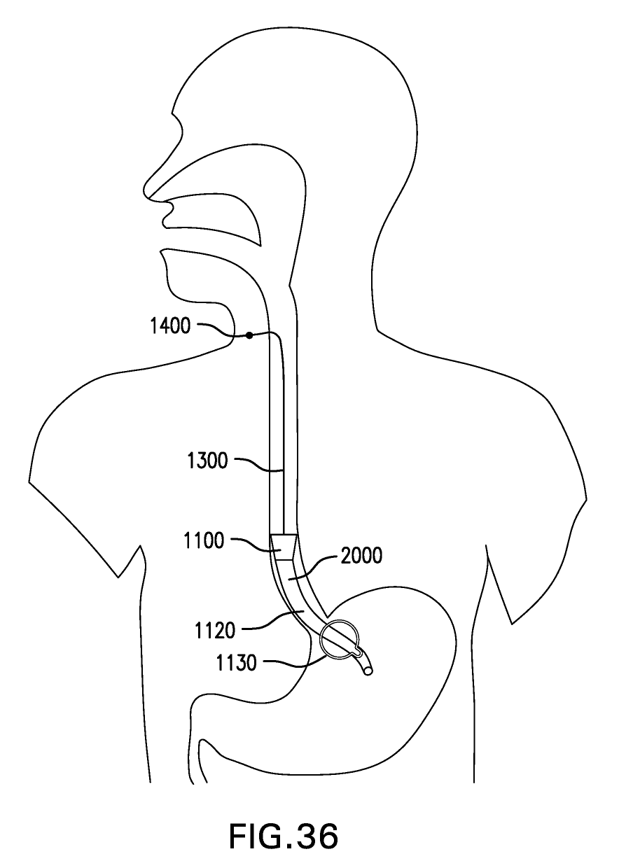

FIG. 36 shows one embodiment of a flow restriction device 2000. Flow restriction device 2000 may restrict a flow of food from the esophagus into the stomach. Flow restriction device 2000 may be similar to gastrointestinal bypass device 1000, but with a sleeve 1120 that is shorter. Flow restriction device 2000 may be maintained in a particular portion of the esophagus and/or stomach by a tether assembly 1300 and a tether anchor 1400.

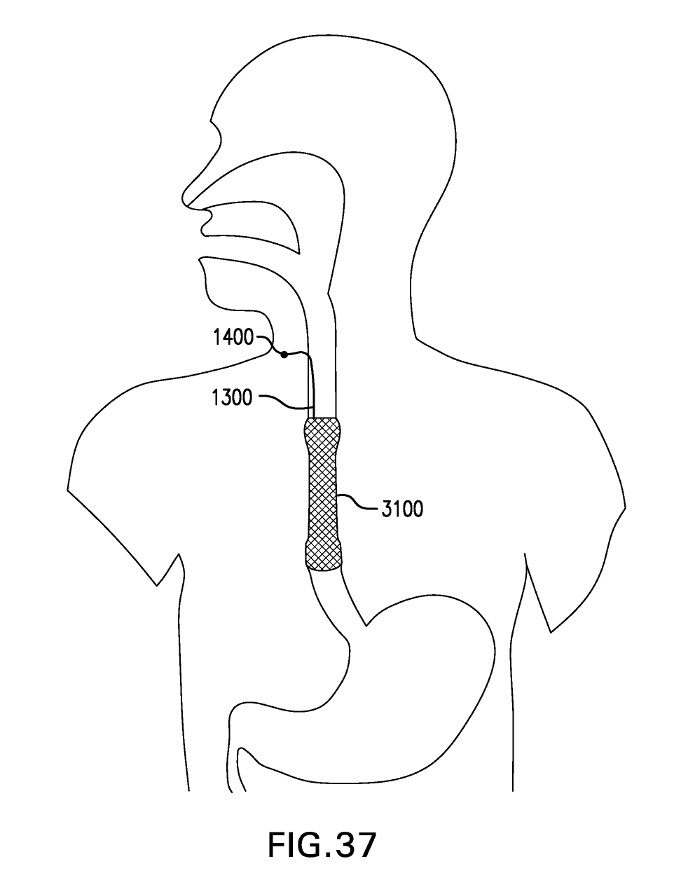

FIG. 37 shows one embodiment of an esophageal stent 3100. Esophageal stent 3100 may be maintained in a particular portion of the esophagus by a tether assembly 1300 and a tether anchor 1400. Esophageal stent 3100 may be coupled to a tether 1310.

In some embodiments, methods of placing a device, such as a bypass tube within the gastrointestinal tract of a patient can involve delivering the bypass tube for implantation within the gastrointestinal tract transorally, while delivering anchoring structures for the device through a different access pathway, such as via a fistula between the esophagus and another body structure, such as the skin or a bony structure for example. The method can include inserting the bypass tube into a mouth of the patient and advancing the device bypass tube into the esophagus; creating a fistula between the esophagus and the skin of the patient; placing a tether through the fistula, the tether having a distal portion coupled to the bypass tube; and securing a proximal portion of the tether to an anchor site. A proximal opening of the bypass tube can be positioned within a gastrointestinal tract lumen, such as the upper esophagus, lower esophagus, GEJ region, or stomach for example without extending through the fistula. In contrast, a portion of the tether can extend through the fistula, and an anchor attached to the proximal end of the tether is spaced proximally apart from the proximal opening and attached to a structure outside of the gastrointestinal tract. Such securement methods can advantageously require no special restrictions on patient bathing and stably secure the device over an extended period of time. A device, e.g., cuff and/or bypass tube can thus advantageously move axially within the esophagus, which can continue to physiologically foreshorten and elongate with the peristaltic wave, e.g., via swallowing.

In some embodiments, methods of securing a device within a gastrointestinal tract lumen of a patient can include inserting a delivery tool (e.g., a balloon catheter for example) including the device into a mouth of the patient, and advancing the delivery tool and the device into the esophagus. A fistula can be created between the esophagus and another anatomical structure, such as the skin of the patient. A tether can be placed through the fistula, the tether having a distal portion coupled to the device. A proximal portion of the tether can be secured to an anchor site spaced apart from the esophagus. The delivery tool can be dissociated from the device, and removed from the patient, leaving the device within the gastrointestinal tract lumen. The device is not placed through, and does not extend through the fistula. The device could include a cuff and/or bypass tube, sensor, valve, artificial stoma or restriction element, electrical or other electromagnetic stimulation systems (including electrodes), intragastric balloons, cameras, reservoirs for delivery of a drug or other therapeutic agent, or other devices as disclosed elsewhere herein for example. A proximal end of the device can be positioned within the upper esophagus, lower esophagus, GEJ region, or stomach for example without extending through the fistula.

While the foregoing has been with reference to particular embodiments of the invention, it will be appreciated by those skilled in the art that changes in these embodiments may be made without departing from the principles and spirit of the invention.

1. A gastrointestinal bypass device, comprising:
a cuff configured to be placed at least partially in an esophagus of the patient;
a sleeve coupled to the cuff, the sleeve configured to be placed at least partially in a stomach of the patient; and
a tether having a distal portion coupled to the cuff and/or the sleeve, the tether configured to be placed through a fistula between an esophagus and a skin of the patient, the tether having a length sufficient to extend from the cuff and/or the sleeve to the skin.

2. The device of claim 1, wherein the tether has a length of 10 cm to 30 cm.

3. The device of claim 1, wherein the tether has a thickness of at least 8 Fr (2.67 mm) where the tether passes through a wall of the esophagus.

4. The device of claim 1, wherein the tether has a bending stiffness configured to hold the tether away from an inside of the esophageal wall where the tether passes through a wall of the esophagus.

5. The device of claim 1, wherein the tether has a bending stiffness configured to not allow the tether to rest against an inside of the esophageal wall immediately below where the tether passes through a wall of the esophagus.

6. The device of claim 1, wherein the tether has an outer surface that is soft.

7. The device of claim 1, wherein the tether has an outer surface with a hardness of Shore 70A to Shore 100A.

8. The device of claim 1, wherein the tether elongates less than 10% under tension.

9. The device of claim 1, wherein the tether includes a tube.

10. The device of claim 9, wherein the tube is reinforced with a mesh and/or a braid.

11. The device of claim 1, wherein the tether includes one or more lumens.

12. The device of claim 1, wherein a proximal portion of the tether is tapered.

13. The device of claim 1, further comprising:
a pull tab coupled to a proximal portion of the tether.

14. The device of claim 1, further comprising:
a tether anchor configured to be coupled to a proximal portion of the tether, the tether anchor configured to secure the proximal portion of the tether to an anchor site.

15. The device of claim 14, wherein the tether anchor is configured to be placed on the skin.

16. The device of claim 15, wherein the tether anchor includes a skin button.

17. The device of claim 14, wherein the tether anchor is configured to be placed under the skin.

18. The device of claim 17, wherein the tether anchor includes one or more barbed sutures.

19. The device of claim 14, wherein the tether anchor is configured to be placed in the fistula.

20. A gastrointestinal bypass device, comprising:
a cuff configured to be placed at least partially in an esophagus of the patient;
a sleeve coupled to the cuff, the sleeve configured to be placed at least partially in a stomach of the patient; and
a tether means coupled to the cuff and/or the sleeve, the tether means configured to maintain the cuff and the sleeve in a particular portion of the gastrointestinal tract.

21-40. (canceled)
